(19) United States
(12) Patent Application Publication
(10) Pub. No.: US 2026/0332583 A1
(43) Pub. Date: Oct. 22, 2026

(54) IMPROVED SECURITY ESTABLISHMENT METHODS AND SYSTEMS

(71) Applicant: KONINKLIJKE PHILIPS N.V., EINDHOVEN (NL)

(72) Inventor: Oscar Garcia Morchon, Eindhoven (NL)

(21) Appl. No.: 19/101,291

(22) PCT Filed: Aug. 4, 2023
(86) PCT No.: PCT/EP2023/071677

§ 371 Date: Feb. 5, 2025

(30) Foreign Application Priority Data
Aug. 12, 2022 (EP) 22190117.6
Aug. 12, 2022 (EP) 22190129.1
Aug. 12, 2022 (EP) 22190133.3
Aug. 12, 2022 (EP) 22190140.8
Aug. 12, 2022 (EP) 22190146.5
Aug. 12, 2022 (EP) 22190152.3
Aug. 12, 2022 (EP) 22190162.2
Aug. 12, 2022 (EP) 22190168.9
Aug. 12, 2022 (EP) 22190179.6
Aug. 12, 2022 (EP) 22190185.3
Aug. 12, 2022 (EP) 22190191.1
Sep. 29, 2022 (EP) 22198612.8
Sep. 29, 2022 (EP) 22198619.3
Sep. 29, 2022 (EP) 22198622.7
Sep. 29, 2022 (EP) 22198629.2
Sep. 29, 2022 (EP) 22198638.3
Sep. 29, 2022 (EP) 22198644.1
Sep. 29, 2022 (EP) 22198647.4
Sep. 29, 2022 (EP) 22198655.7
Sep. 29, 2022 (EP) 22198659.9
Sep. 29, 2022 (EP) 22198663.1
Sep. 29, 2022 (EP) 22198668.0
Jan. 6, 2023 (EP) 23150571.0
Feb. 10, 2023 (EP) 23156019.4
Apr. 12, 2023 (EP) 23167539.8
May 11, 2023 (EP) 23172719.9
May 25, 2023 (EP) 23175238.7
Jun. 6, 2023 (EP) 23177737.6
Jul. 28, 2023 (EP) 23188517.9

(51) Int. Cl. H04L 9/40

(52) U.S. Cl. CPC H04L 63/0869; H04L 63/083

(57) ABSTRACT

The invention relates to methods and devices for setting up a secure communication channel with an improved key exchange for a security establishment protocol or procedure.

### FIELD OF THE INVENTION

The invention relates to security establishment procedures between devices and/or applications in a wired or wireless data network, such as-but not limited to-password authenticated key exchange (PAKE).

In another embodiment, one or more network functions (NFs) in the CN is in charge of monitoring whether security procedures based on a security procedure.

### BACKGROUND OF THE INVENTION

A password or a passphrase is a string of characters that is usually chosen by a user. Passwords are often used to authenticate a user to allow access to a resource. Since most user-chosen passwords have low entropy and weak randomness properties, these passwords may not be used directly as cryptographic keys.

Key derivation functions (KDFs) are deterministic algorithms that are used to derive one or more secret keys (cryptographic keying material) from a secret value, such as a main key, a password, or a passphrase using a pseudorandom function (PRF) which typically uses a cryptographic hash function or block cipher. A password based KDF (PBKDF) may be defined by the choice of the PRF and a fixed iteration count C. An input to an execution of a PBKDF may include a password P, a salt S, and an indication of the desired length kLen of a desired master key mk in bits, denoted as kLen. Symbolically, this can be expressed as follows:

mk=PBKDF_{(PRF, C)}(P, S, kLen).

The salt may be a binary value that is used as an input to the PBKDF to allow generation of a large set of keys for a given password. The iteration count (C) is a fixed value that determines how many times the PRF iterates to generate one block of the master key. The iteration count may be selected as large as possible, as long as the time required to generate the key using the entered password is acceptable for users.

Modern PBKDFs, such as PBKDF2 (specified in IETF specification RFC 2898), can be based on a recognized cryptographic hash, such as SHA-2, use more salt (at least 64 bits and chosen randomly) and a high iteration count.

FIG. 1 shows an example of a PBKDF2 algorithm for the derivation of master keys from passwords, which uses a keyed-hash message authentication code or hash-based message authentication code (HMAC) with Secured Hash Algorithm 1 (SHA-1) as PRF. The digest size of the hash function in bits is denoted as hLen. HMAC is a specific type of MAC involving a cryptographic hash function and a secret cryptographic key. As with any MAC, it may be used to simultaneously verify both the data integrity and authenticity of a message.

The input of the algorithm of FIG. 1 comprises a password P, a salt S, an iteration count C, and a desired length kLen of a master key in bits (at most (232-1)×hLen). Parameters of the algorithm are the PRF (which is a HMAC with an approved hash function) and a digest size hlen of the hash function. Output of the algorithm is the master key mk.

First, it is checked whether the desired length of the master key is largen than the maximum length. If so, then an error indicator (Err-i) is returned and the procedure stops.

If the desired length is within the acceptable range, a loop parameter len for an outer loop (i=1 to len) for code portions Ti to be combined is determined based on the ratio kLen/hLen and each code portion is calculated in an inner loop (j=1 to C).

FIG. 2 shows a signaling diagram of a message exchange between two devices A and B that includes a specific augmented PAKE process (called “SPAKE2+”) as described in Taubert, T. et al.: “SPAKE2+, an Augmented PAKE (Draft)”, IETF, 5 May 2022. A similar message exchange applies to a specific balanced PAKE process denoted SPAKE2 as described in W. Ladd et al.: “SPAKE2, a PAKE (Draft)”, IETF, Jun. 2, 2021.

SPAKE2+ defines a PAKE that allows two parties (a party may be a device, an application etc.) A and B to agree on a common symmetric key in an authenticated manner, where the authentication is based on a (weak) password. After an initial preamble exchange (pre) to communicate identities, protocol version, PBKDF parameters, etc., the procedure executes the PAKE involving four messages and two round trips. The first party A computes a shared secret pA (cpt pA) and the second party B computes a shared secret pB (cpt pB). The first two messages (including the respective shared secrets pA and pB) are used for setting up a protocol (s-u prot) and to agree on a common secret. Then, the second party B computes a confirmation cB (cpt cB) and the first party computes a confirmation cA (cpt cA). The last two messages (including the respective confirmations cB and cA) are used for deriving secrets (der scr) and for key confirmation. Two of the messages may be combined into one (pB+cB), as they are successively transmitted into the same direction (from B to A).

However, in the context of SPAKE2+ or other security establishment procedures, it is desirable to improve security establishment procedures thereby to further enhance security.

### SUMMARY OF THE INVENTION

It is an object of the present invention to provide enhanced security for communication between devices and/or applications.

This object is achieved by apparatuses, communication devices, equipment and systems, methods and computer program products according to the appended claims.

Thus, security establishment messages (e.g., PAKE-type or other key exchange/security establishment messages) between two specific communication devices or applications can be associated to a given communication link e.g. by a session identifier, while the session identifiers of the communicating devices for the same security establishment process do not need to be equal. This can be advantageous when the security establishment is executed at the application layer, because in this case the security establishment protocol is not aware of underlying networking identifiers (e.g., MAC or IP addresses).

Thus, when the communicating devices exchange one or more initial messages (e.g., preamble messages), it can be ensured that both devices have a single security establishment session alive, or if they have multiple sessions alive, that they are linked to the correct peer devices.

In addition, the security establishment messages can be rearranged to require only two round trips in total. This is advantageous to achieve a more efficient protocol by decreasing protocol latency and thus increasing user experience. This can also be useful in cases where the protocol is to be executed over a constrained network and/or with many devices simultaneously.

Accordingly, security establishment messages (e.g., PAKE-type or other key exchange messages) between two specific communication devices or applications can be associated to a given communication link e.g. by a session identifier, while the session identifiers of the communicating devices for the same security establishment process do not need to be equal. This is advantageous when the security establishment is executed at the application layer, because in this case the security establishment protocol is not aware of underlying networking identifiers (e.g., MAC or IP addresses).

When a communication device is allowed to have a single or at least limited number of key exchange alive sessions at any point of time, the communication device is enabled to limit the number of incoming requests. This also ensures that an attacker (man-in-the-middle) cannot inject many potential preamble messages exhausting the available sessions.

According to a first aspect (e.g., related to one end of a transmission link), an apparatus is provided for controlling a security establishing protocol (process) between a first communication device and a second communication device over the transmission link, wherein the apparatus is adapted to: receive from the first communication device a preamble message including a security establishment identifier; and
allocate the security establishment identifier received from the first communication device as peer session identifier for subsequent messages;
wherein the apparatus is further adapted to perform at least one action selected from the group of: limiting the number of first preamble messages allowed per unit of time;
limiting the number of allocated peer session identifiers; and
discarding an allocated peer session identifier if a subsequent first security establishment message is not received within a predetermined time period.

According to a second aspect, a communication device comprising the apparatus of the first aspect is provided.

According to a third aspect, a method of controlling a security establishment process between a first communication device and a communication device over a transmission link is provided, wherein the method comprises:
receiving from the first communication device a preamble message including a security establishment identifier; and
allocating the security establishment identifier received from the first communication device as peer session identifier for subsequent messages;
wherein the method further comprises a selection of at least one from the group of:
limiting the number of first preamble messages allowed per unit of time;
limiting the number of allocated peer session identifiers; and
discarding an allocated peer session identifier if a subsequent first security establishment message is not received within a predetermined time period.

According to a fourth aspect, a computer program product is provided, which comprises code means for producing the steps of the above method according to the third aspect when run on a computer device.

Finally, according to a fifth aspect, a system comprising two or more communication devices including a communication device of the second aspect is provided.

Accordingly, security establishment messages (e.g., PAKE-type or other key exchange messages) between two specific communication devices or applications can be associated to a given communication link e.g. by a session identifier, while the session identifiers of the communicating devices for the same security establishment process do not need to be equal. This is advantageous when the security establishment is executed at the application layer, because in this case the security establishment protocol is not aware of underlying networking identifiers (e.g., MAC or IP addresses). When a communication device is allowed to have a single or at least limited number of key exchange alive sessions at any point of time, the communication device is enabled to limit the number of incoming requests. This also ensures that an attacker (man-in-the-middle) cannot inject many potential preamble messages exhausting the available sessions.

According to a first option which may be combined with any of the above first to fifth aspects, the first communication device may comprise a commissioning tool configured to use the security establishment process to interact with the second communication device in order to perform at least one selected from the group of commissioning, configuring, authenticating and authorizing. Thereby, the proposed security enhancement can be applied for key exchange in connection with a commissioning process.

According to a second option which can be combined with the first option or any of the above first to fifth aspects, the second communication device may be comprised in a medical device or a personal healthcare device or a smart home device. Thus, the proposed security enhancement can be applied for various medical, healthcare and smart home applications.

According to a third option which can be combined with the first or second option or any of the above first to fifth aspects, the apparatus may be configured to run a password authenticated key exchange protocol to mutually authenticate the first and second communication devices to each other and establish a secret. Thereby, the proposed security enhancement can be applied in connection with a password authenticated key exchange protocol.

According to a fourth option which can be combined with any of the first to third options or any of the above first to fifth aspects, preamble messages may be exchanged between the first and second communication devices or between the first communication device or the second communication device and a relay device in an initial device discovery phase. Thereby, the initial device discovery phase between the related communication devices or via a relay device can be used for exchanging the preamble messages of the proposed security enhancement.

According to a fifth option which can be combined with any of the first to fourth options or any of the above first to fifth aspects, a single preamble message may be exchanged between the first and second communication devices. Thereby, the proposed security enhancement protocol or procedure can be simplified in cases where it is known that multiple communication devices are not to be expected at one communication end.

According to a sixth option which can be combined with any of the first to fifth options or any of the above first to fifth aspects, a content of a preamble message may be included in a discovery message, wherein the first security establishment message may be included in a direct communication request message. Thus, the proposed security enhancement protocol or procedure can be integrated in a device discovery signaling process.

According to a seventh option which can be combined with any of the first to sixth options or any of the above first to fifth aspects, a password authenticated key exchange protocol may be run to mutually authenticate the first and second communication devices to each other and establish a secret over a first wireless communication protocol and authorize the first communication device to access communication resources of a second communication link. In an example, the second communication link may use a cellular communication protocol. Thereby, the proposed procedure can be used to provide access to an additional (e.g., at least partially cellular) communication link.

It is noted that the above apparatuses may be implemented based on discrete hardware circuitries with discrete hardware components, integrated chips, or arrangements of chip modules, or based on signal processing devices or chips controlled by software routines or programs stored in memories, written on a computer readable media, or downloaded from a network, such as the Internet.

It shall be understood that the apparatuses, communication device, methods, the computer program product and system may have similar and/or identical preferred embodiments, in particular, as defined in the dependent claims.

It shall be understood that a preferred embodiment of the invention can also be any combination of the dependent claims or above embodiments with the respective independent claim.

These and other aspects of the invention will be apparent from and elucidated with reference to the embodiments described hereinafter.

### BRIEF DESCRIPTION OF THE DRAWINGS

In the following drawings:

FIG. 1 shows an algorithm for calculating a master key using a PBKDF;

FIG. 2 shows a signaling diagram that includes a message exchange according to a security establishment protocol, e.g., SPAKE2+or SPAKE2 process;

FIG. 3 schematically shows a signaling and processing diagram for an improved security establishment protocol according to various embodiments;

FIG. 4 schematically shows a block diagram of a communication device according to various embodiments;

FIG. 5 schematically shows a block diagram of a PBKDF configurator element or function according to an embodiment;

FIG. 6 schematically shows a block diagram of an implicit PBKDF parameter generator element or function according to an embodiment;

FIG. 7 shows a flow diagram of a conditional PBKDF parameter exchange according to an embodiment;

FIG. 8 shows a signaling diagram of a preamble exchange with PAKE support information according to an embodiment;

FIG. 9 shows a signaling diagram of a preamble exchange with hash function information according to an embodiment;

FIG. 10 shows a signaling diagram of a preamble exchange with reliability information according to an embodiment;

FIG. 11 schematically shows a block diagram of a lower layer protocol support for fragmentation handling according to an embodiment;

FIG. 12 shows a flow diagram of a key derivation procedure based on exchanged preamble information according to an embodiment;

FIG. 13 schematically shows an exemplary protocol stack according to an embodiment;

FIG. 14 schematically shows a signaling and processing diagram for an improved security establishment process, e.g., based on SPAKE2(+), with reduced number of roundtrips according to an embodiment.

FIG. 15 schematically shows a signaling and processing diagram for UE-to-UE relay scenarios according to an embodiment;

FIG. 16 schematically shows a signaling and processing diagram for personal IoT network scenarios according to an embodiment;

FIG. 17 schematically shows a signaling and processing diagram for UE-to-UE relay scenarios according to an embodiment;

FIG. 17b schematically shows a signaling and processing diagram for UE-to-UE relay scenarios according to an embodiment;

FIG. 18 schematically shows a signaling and processing diagram for UE-to-UE relay scenarios according to an embodiment;

FIG. 19 schematically shows elements and interfaces in a personal IoT network scenario;

FIG. 20 schematically shows a signaling and processing diagram for personal IoT network scenarios according to an embodiment;

FIG. 21 schematically shows a signaling and processing diagram for UE-to-UE relay scenarios according to an embodiment, and

FIG. 22 schematically shows a signaling and processing diagram for UE-to-UE relay scenarios according to an embodiment.

### DETAILED DESCRIPTION OF THE EMBODIMENTS

Embodiments of the present invention are now described based on various modifications of security establishment procedures or protocols (SEP) relying on, e.g., a generic PAKE or SPAKE2 or SPAKE2+ or others, where the protocol allows establishing a security property, such as for example at least one of authorization, mutual authentication based on a shared password, key establishment based on a public key exchange, and key confirmation.

The inventor has realized that real-world implementations and certain use-cases may give rise to a need to better secure such key exchanges. Wireless networks are vulnerable to such things as man-in the middle attacks. Thus, the inventor has realized that that key exchange messages can be better secured by providing at least one of a binding to a specific set of communication parties and/or to a specific key exchange procedure. Likewise, it has been determined that they may be benefit from an improved derivation of one or more key derivation parameters based on one or more exchanged parameters, and/or a support for a potential key exchange upgrade to enhance security (e.g., due to increased processing power achieved by e.g. a quantum computer). Since communications are vulnerable to interception and are time-sensitive since a user may be involved, they may benefit from a reduction of the number of round trips of the key exchange process. Also means of providing authentication and authorization, or a reliable transfer (e.g., retransmissions) if messages of a PAKE protocol are transported over a non-reliable channel (e.g., a channel via a relay device (e.g., UE-to-UE relay—a use-case that the inventor has determined as exhibiting particular vulnerability) or between personal IoT networks (PIN) may be of benefit.

In the following, multiple modifications or enhancements when using an SEP are described based on embodiments where a first device or application uses PAKE to configure a second device or application. The following embodiments relate to enhancements in connection with provision of details on the preamble, details on how the exchange parameters in the preamble can be used in the PBKDF, details on negotiation of a PAKE in the preamble, alternative options for the interaction (e.g., selection of the device that starts PAKE communication) after preamble messages exhibit different features, and reduction of the number of round trips.

The proposed enhanced SEP procedure may be applied in connection with wired or wireless transmission channels or communication streams over communication networks and possibly other networks. In the 3GPP specifications 23.303, 23.304, 24.334 and 24.554 for 4G and 5G networks, respectively, so-called proximity service (ProSe) functions are defined to enable—amongst others connectivity for cellular communication devices (e.g., UEs) that are temporarily not in coverage of an access device (e.g., eNB). This particular function is called ProSe UE-to-network relay, or Relay UE. The Relay UE is a communication device that helps another out-of-coverage (OoC) UE to communicate to the eNB (i.e., access device) by relaying application and network data traffic in two directions between the OoC UE and the eNB. The local communication between the Relay UE and the OoC-UE is called D2D communication or Sidelink communication or PC5 communication. The abbreviation “PC5” designates an interface for sidelink communication as defined by ProSe. Furthermore, the abbreviation “UL” is used for the uplink direction from the communication device (e.g., UE) to the access device (e.g., eNB, gNB), the abbreviation “DL” for the downlink direction from the access device (e.g., eNB, gNB) to the communication device (e.g., UE), and the abbreviation “SL” for sidelink communication between two or more communication devices (e.g. UEs).

Furthermore, 3GPP specifications TR 23.733 v 15.1.0 and TR 36.746 v 15.1.1 provide studies on architectural enhancements e.g. to enable an Internet of Things (IOT) device (in a role of Remote UE) to operate on very low power by using a Relay UE to connect to the wider network. Because the Relay UE is physically very close, it can be reached using very low power transmissions. This also includes security, speed and stability improvements to ProSe. These extensions of ProSe are called enhanced ProSe (“eProSe”).

ProSe can also be used for direct communication between two UEs. Additional radio level details on ProSe, V2X and sidelink communication can be found in 3GPP specifications TR 37.985, TS 38.300 and TR 38.836.

There are IoT networks where devices communicate with each other and with other networks via a gateway. These networks may be termed personal IoT networks (PINs) and the devices in such a network may be termed ‘PIN elements’. Such networks may communicate with the other network via a PIN element with gateway capability (PEGC). The PIN elements of a PIN may be managed by at least one PIN element with management capability (PEMC). An example of the other network may be a 5G network and document TR 23.700-18 describes enhancements of 5G systems to support PINs in accordance with the service requirements as documented in TS 22.261.

According to TR 23.700-88 (for example), a PIN element may be a UE or non-3GPP device that can communicate within a PIN (via a PIN direct connection, via a PEGC, or via a PEGC and a 5G capability (5GC)), or outside the PIN via a PEGC and a 5GC. According to TR 23.700-88, a PIN element with gateway capability is a PIN element with an ability to provide connectivity to and from a 5G network for other PIN elements, or to act as relay for a communication between PIN elements. A PIN element with management capability is a PIN element with capability to manage the PIN. According to TR 23.700-88, a PIN direct connection refers to the connection between two PIN elements without PEGC, any 3GPP RAN or core network entity in the middle. Multiple key issues are included in TR 23.700-88 including 5GC architecture enhancements to support PIN, PIN and PIN element discovery and selection, management of PIN and PIN elements, communication of PIN, authorization of PIN, policy and parameters provisioning for PIN, or identification of PIN and PIN elements. TR 23.700-88 also includes multiple feasible solutions that illustrate such a protocol layer structure as shown in FIG. 13. It should be understood that the layer structure of FIG. 13 is not limited to 5G networks and many other networks may employ a similar layer structure.

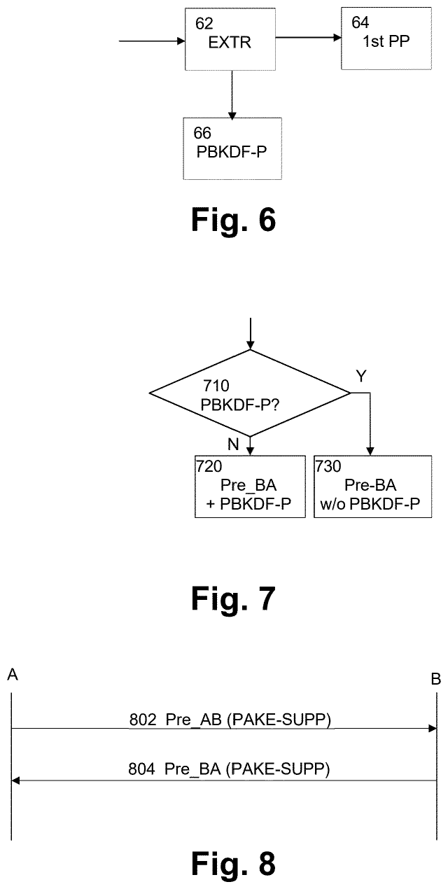

FIG. 13 schematically shows an exemplary protocol stack according to an embodiment that may be applied to e.g. a system as described in TR 23.700-88 with reference to its FIG. 6.0B.2-2.

As shown in FIG. 13, a PIN layer (responsible for processing PIN IDs, PIN elements, PEGCs and/or PEMCs) may be configured to run over multiple transport (TRA) and/or physical (PHY) layers (including WIFI, Bluetooth, 5G ProSe, etc.) and support a a higher application (APP) layer (which may be configured to control light bulbs, power sockets, washing machines or any other controllable devices) in handling key exchange and/or other security establishment functions as described in the following embodiments.

In an example, a supported PIN element function (S-PEF) may represent functionalities providing communication within the PIN layer (e.g., via a PIN direct connection or via a PEGC), or outside the PIN (e.g., via a PEGC). The PEF is also able to communicate with a PEMC for been configured, for discovery and for authentication and authorisation.

A non-supported PEF (NS-PEF) may represent functionalities that communicate directly between the transport (TRA) and/or physical (PHY) layers and the higher application layer without using the intermediate PIN layer.

Furthermore, there may be key issues such as protection of an identification of the PIN and PIN privacy, a secure communication between PINEs, secure policy and parameter provisioning, authorization of a PIN element (PINE), PIN and PINE discovery authorization, controlling access of PIN elements to the other (e.g., 5G) network, authentication and authorization to a PINE, secure authentication of PINE, or secure provisioning of credentials in non-3GPP device via PEGC.

There may be situations involving a relay device such as a UE-to-UE relay, i.e., a first device A talking to a second device B over a third device acting as relay. In such a use case, there may be issues including privacy protection, integrity protection, confidentiality protection or authorization over the UE-to-UE relay. The function of a relay may also be seen in a mesh network.

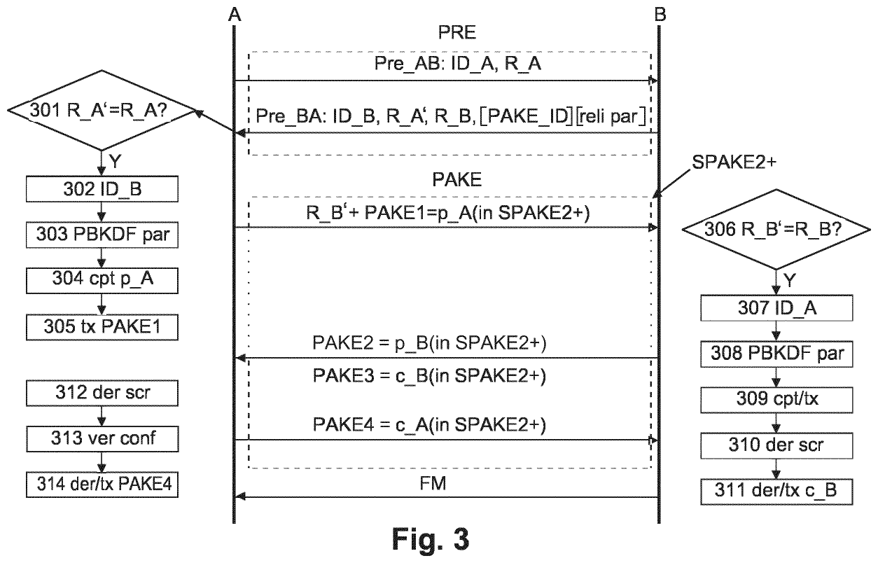

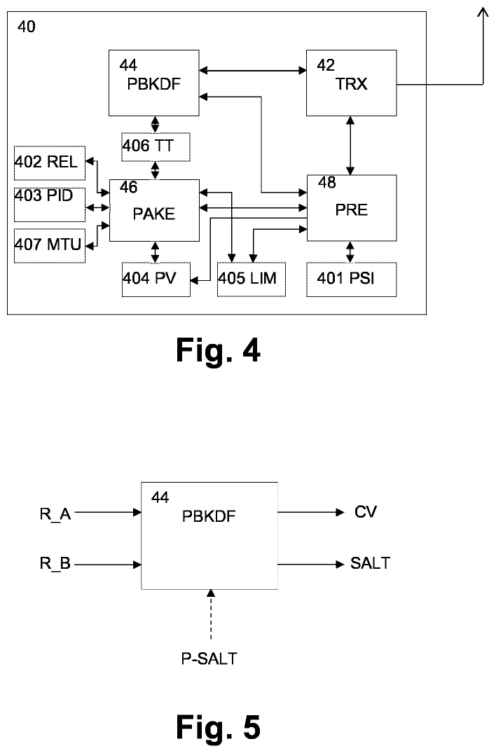

In the following, various embodiments with improvements of SEPs are described. Further details of their involved hardware components are described later with reference to FIG. 4, while details of their procedural and signaling steps are described with reference to FIGS. 3 and 5 to 13.

Some embodiments are summarized in FIG. 3 where a first device or device A wishes to setup a secure communication channel with a second device or device B.

FIG. 3 schematically shows a signaling and processing diagram for an improved SPAKE-based SEP according to various embodiments. In this diagram, exchange of information and its direction between the two devices A and B is indicated by a corresponding arrow and processing steps are indicated by respective blocks, while the time proceeds from the top to the bottom of FIG. 3. Not all the steps may always be required or applied and some steps may be executed multiple times for increased accuracy or continuous processes.

The first device A may be a mobile application used to configure a second device B, e.g., a medical device or a personal healthcare device or a smart home device. The first device may also be a PEMC or a PEGC. Device A and device B may communicate over WiFi, or other communication means such as Bluetooth Low Energy (BLE), thread, or over a 3GPP standard, e.g., using Sidelink, or over a low power communication protocol.

It may be the case that a device (or a combination of devices) is used to configure other devices, particularly for connection to a given network. The device (or devices) may be dedicated for this purpose or it (they) may be more general-purpose device(s) with a function running on it (them). An example of a function running on a device may be an application (or ‘app’) running on a smartphone. An example of a multiple device providing the function may be one device relaying the messages used for the configuring to another device such as a server. The term ‘commissioning tool’ may be used to refer to the function provided by the device (or combination of devices) performing the task of configuring or it may be used to refer to the device(s) themselves.

The two devices A and B may need to run a PAKE such as an augmented or balanced PAKE (e.g., SPAKE2+or SPAKE2, respectively) to mutually authenticate to each other and establish a secret. However, there may be potentially many pairs of devices A and B doing the same at the same time. This may occur, e.g., when a commissioning tool (such as device A, when providing a function for this purpose) for the IoT network needs to commission many devices B, or when a device B may be configured by multiple commissioning tools.

Thus, it is important to associate the PAKE exchange to a given communication link between two specific devices. Each device A or B may associate such PAKE exchange to a given communication (link) identified by a given a communication session identifier. The session identifiers of devices A and B for the same PAKE exchange do not need to be equal. This may be important when the PAKE process is executed at the application protocol layer because in this case the PAKE is not aware of underlying networking identifiers such as Media Access Control (MAC) addresses or IP addresses of the lower protocol layer.

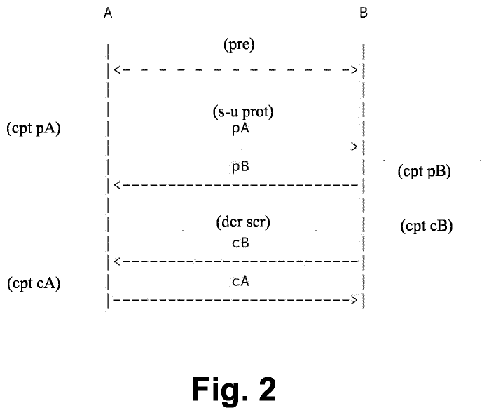

When devices A and B exchange some initial messages (e.g., preambles as described in FIG. 2), devices A and B may need to ensure that they have a single PAKE session alive, or if they have multiple, that they are linked to the correct peers.

In an example, device A may be a commissioning tool that may interact with many potential devices to be commissioned and/or configured and/or authenticated and/or authorized and/or security configured, for instance, smart home devices.

In an example, device B may be a smart home device that may have to deal with multiple commissioning devices A (e.g., an app running on a mobile phone) at the same time when multiple users or multiple apps are trying to connect to it.

According to various embodiments, two preamble messages Pre_AB (transmitted from device A to device B) and Pre_BA (transmitted from device A to device B) are proposed to be exchanged during the preamble phase (PRE).

In message Pre_AB, device A can send to device B its short (e.g., 8 bits) PAKE identifier ID_A. As an addition option, device A may also include in the message Pre_AB a (long) random value R_A.

In message Pre_BA, device B can send to device A its short (e.g., 8 bits) PAKE identifier ID_B. As an addition option, device B may also include a (long) random value R_B and a previously received R_A′. As further option, described in later embodiments, device B may further include in its message Pre_BA at least one of an identifier (PAKE_ID) of the PAKE protocol version and a reliability parameter (reli par).

Upon reception of the Pre_BA message, device A checks in step 301 whether R_A′ is equal to R_A, and if it is, then the device A sets the peer session identifier to be used in subsequent messages as ID_B (step 302). If R_A′ and R_A do not match, device A may abort the process, e.g., with an error message or a report (e.g., to identify man-in-the-middle attacks or other attacks) or drop the executed protocol or restart the protocol.

Then, the PAKE phase starts (which may be a SPAKE2+ process), where during a first round, device A sends, in a first message PAKE1, its public share p_A to device B which in turn responds with its own public share secret p_B in a second message PAKE2. For the purposes of the present, a public share, e.g., p_A or p_B, is a value transmitted by a transmitting entity that allows the receiving party to derive a shared secret with the transmitting entity by combining the received public share with a private value only known to the receiving party, e.g., as defined in SPAKE2+. In a second round, both parties may use a unique and secret protocol transcript (TT) to derive a shared symmetric secret (shared key) from the protocol transcript. The key confirmations c_A and c_B are derived from the shared symmetric secret and exchanged in third and fourth messages PAKE3, PAKE4, as initially described in connection with FIG. 2.

More specifically, in step 303 of FIG. 3, device A determines or applies PBKDF parameters based on, e.g., information provided in respective fields exchanged with the preamble messages or as provided/configured by a central managing entity. Then, it computes in step S304 the common secret p_A and transmits it in step 305 in the first message PAKE1 together with the latest received R_B′.

Upon reception of R_B′, device B can check in step 306 whether R_B′ is equal to R_B, and if it is, then device B sets in step 307 the peer session identifier to be used in subsequent messages as ID_A. If R_B′ and R_B are not equal, device B may abort the process, e.g., with an error message or a report (e.g., to identify man-in-the-middle attacks or other attacks), or drop the executed protocol or restart the protocol.

In step 308, device B may determine or apply the PBKDF parameters based on, e.g., information provided in respective fields exchanged with the preamble messages or as provided/configured by a central managing entity. Then, it computes and sends the common secret p_B in the second message PAKE2 to device A in step 309. Additionally, device B derives the shared secret in step 310 based on the received common secret p_A. Then, it derives and sends the key confirmation c_B in step 311 in the third message PAKE3 to device A.

In step 312, device A derives the shared secret based on the common secret p_B received with the second message PAKE2. Then, it verifies the key confirmation c_B received in the third message PAKE3 in step 313 and derives the key confirmation c_A and sends it in step 314 to device B in the fourth message PAKE4.

The key confirmations c_A and c_B in the last round-trip may be authentication keys derived from the shared symmetric secret. In an example, both devices A and B may be configured to compute an authentication tag with an authentication key produced over the protocol transcript.

Finally, the final message (FM) is transmitted from device B to the initiating device A to close the procedure.

In some scenarios addressed e.g. in TR 33.740, the exchange of the preamble messages or some of its fields may be done in an initial discovery phase between UEs through a UE relay or between UE and a UE relay, e.g., a discovery phase associated to UE-to-UE relay scenarios. In such scenarios, the PAKE exchange may be done during the establishment of a secure PC5 interface taking place after the discovery phase. In some scenarios addressed in TR 33.882, the exchange of the preamble messages may be done between in a discovery phase of a PINE.

Thus, by providing the preamble messages Pre_AB and Pre_BA with PAKE identifiers ID_A and ID_B and random values R_A and R_B, respectively, PAKE sessions can be discriminated to ensured that devices A and B have a single PAKE session alive, or if they have multiple, that they are linked to the correct peers.

In above use case of FIG. 3, a commissioning tool (device A) may need to interact with many devices at the same time. However, device B may be allowed to interact with a single commissioning tool at a time. Thus, device B may have a single PAKE exchange process alive at any point of time.

Therefore, in an alternative embodiment, the steps of FIG. 3 involving R_B are removed since device B can expect that a single device A is active at any point of time, e.g., when device A is a commissioning tool such as an app running on a mobile phone and device B is a smart home device. In such a situation, the exchange may not require the exchange of R_B at all.

Similarly, in an alternative embodiment, instead of requiring the exchange of two preamble messages Pre_AB and Pre_BA, a single preamble message, e.g., Pre_AB or Pre_BA may be involved. For instance, upon reception of Pre_AB, device B would reply with the first PAKE message. In such an alternative embodiment, Pre_AB may only include certain configuration parameters or preferences. In a specific application of this alternative embodiment related to 3GPP proximity services, e.g., for use cases and requirements as in TR 33.740, the content in the Pre_AB message may be included in a model A discovery message, i.e., in an announcing message sent by an announcing UE. Upon reception of the Pre_AB message, i.e., of the announcing discovery message, by device B, i.e., by a monitoring device, device B would reply with a direct communication request (DCR) message initiating the PAKE and including the first PAKE message.

FIG. 4 schematically shows a block diagram of a communication device 40 according to various embodiments, which may correspond to at least one of the above devices A and B (including applications).

The communication device 40 comprises a transceiver (TRX) 42 (e.g., a radio frequency (RF) front end or any other communication unit) for transmitting and receiving messages over a communication channel or link or the like of a wired or wireless network.

A preamble control unit or function (PRE) 48 is provided for generating and decoding preamble messages (e.g., Pre_AB, Pre_BA in FIG. 3) transmitted resp. received by the transceiver 42 during a preamble phase of a PAKE process.

Furthermore, the communication device 40 comprises a PAKE control unit or function 46 for generating and decoding PAKE messages (e.g., PAKE1 to PAKE4 in FIG. 3) transmitted resp. received by the transceiver 42 during a preamble phase of a PAKE process.

Additionally, the communication device 40 comprises a PBKDF control unit or function 44 for deriving keys to be used in the PAKE process.

It should be noted that at least some of the preamble control unit or function 48, the PAKE control unit or function 46 and the PBKDF control unit or function may be combined in a single control unit (e.g., a software-controlled processor or computer) and may be implemented by respective software routines controlling a processor or computer device.

It should be further noted that the dashed blocks of FIG. 4 represent optional components which will be described in more detail in subsequent embodiments.

The preamble control unit or function 48 may be configured to set and/or derive at least one of a peer session identifier (PSI) 401, a session limitation parameter (LIM) 405, a PAKE protocol version identifier (PV) 404, PBKDF configuration parameters, and PAKE configuration parameters for/from transmitted/received preamble messages.

As already mentioned above, a unique and secret protocol transcript (TT) 406 may be used to derive a shared symmetric secret (shared key). To achieve this, the PAKE control unit or function 46 and/or the PBKDF control unit or function 44 may have access to the protocol transcript 406.

Furthermore, reliability settings (REL) 402 may be stored in the communication device 40, which influence the communication behavior of the communication device 40, as described later.

Additionally, as already mentioned above, a PAKE ID (PID) 403 and/or a maximum transmission unit (MTU) 407 may be stored or set in the communication device 40, as described later.

In the above use case of FIG. 3, when device B is allowed to have a limited number of PAKE alive sessions at any point of time, device B may need to limit the number of incoming requests.

Thus, if device B sets the peer session identifier 401 to be used in subsequent messages as ID_A already upon the reception of preamble message Pre_AB (e.g., since R_B is not used) and before the first PAKE message (e.g., the first message of a SPAKE2+ process) is received, then device B may be configured to limit the number of preamble messages Pre_AB allowed per unit of time, and/or to limit the maximum number of allocated peer ID sessions, and/or to discard an allocated ID_A if the first PAKE message PAKE1 is not received before a predetermined timeout. This may be particularly advantageous where R_B is not used—or at least not checked.

The respective parameters (e.g., maximum number of preamble messages per unit of time, maximum number of allocated peer ID sessions, and/or predetermined timeout) may be stored as the session limitation parameter(s) 405 accessible by the preamble control unit of function 48 and/or the PAKE control unit or function 46.

The above limitation settings are advantageous in that an attacker may otherwise inject many potential preamble messages Pre_AB exhausting the available sessions.

FIG. 5 schematically shows a block diagram of a PBKDF configurator element or function according to an embodiment.

The PBKDF configurator element or function may be implemented by the PBKDF control unit or function of FIG. 4.

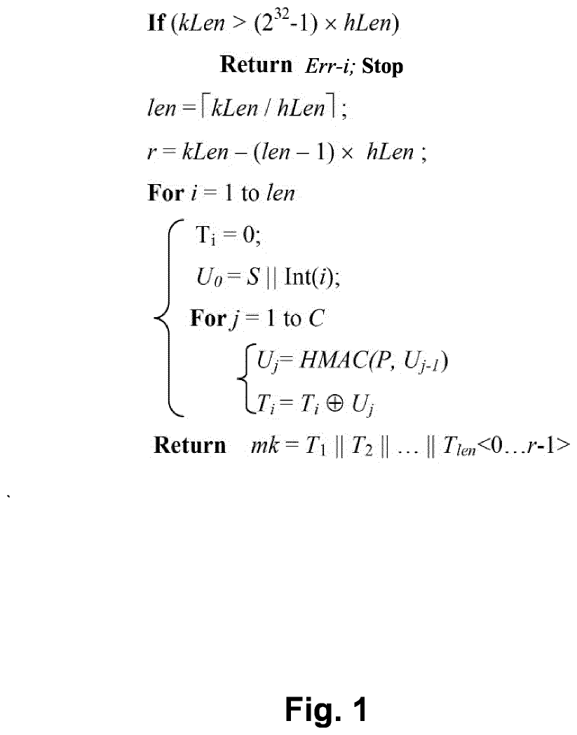

As initially described in connection with FIG. 1, the PBKDF may require several parameters such as a counter value or a salt value. These parameters may be configured, e.g., out-of-band (i.e., not included in the exchanged preamble and PAKE messages), by a default setting, by a manual user operation, or may be specified in a specification. However, in this case, these parameters are rather static. This may raise a problem in that the output of the PBKDF will always be the same after the execution of the initial preamble phase. However, in certain cases it may be preferred to bind a given preamble execution to a subsequent PAKE execution.

In an embodiment, to address this issue, information exchanged in the preamble phase may be used as input to the PBKDF (i.e., to the PBKDF control unit or function 46 in FIG. 4).

More specifically, the PBKDF control unit or function 46 of the communication device 40 (e.g., device A and/or B) may be configured to determine PBKDF parameters (as defined e.g. in Meltem Sönmez Turan et al.: “Recommendation for Password-Based Key Derivation, Part 1: Storage Applications”, NIST Special Publication 800-132, December 2010), e.g., the count value or the salt value, as a function of exchanged parameters in the preamble messages (e.g., Pre_AB, Pre_BA).

It is to be noted that the same embodiment may be applicable if/when a different routine (e.g., a hash function or an HMAC) for deriving a key based on a password (i.e., different than PBKDF) is used.

As indicated in FIG. 5, the exchanged random parameters R_A and R_B (or at least one of these) may be supplied as input values to the PBKDF control unit or function 44 and may be processed (e.g., logically or arithmetically combined) to obtain at least one of the PBKDF parameters count value (CV) and salt value (SALT).

In a first example, the salt parameter may be computed as the logical XOR combination of the exchanged R_A and R_B parameters.

In a second example, the salt parameter may be computed as the logical XOR combination of the exchanged R_A and R_B parameters and a preconfigured salt (P-SALT) set and stored in the communication device 40.

In a third example, the salt parameter may be computed as a hash function of one of the above salt parameter values.

In a fourth example, the salt parameter may be computed as a cryptographic function, e.g., a hash function, of a function, e.g., a concatenation, of the exchanged R_A and R_B parameter.

In a fifth example, the salt parameter may be a subset of bits (e.g., truncation) of any of the above salt parameter values.

In a sixth example, the count value may be set to a minimum value of e.g., 1000 (e.g., as defined in a standard), but dependent on the exchanged parameters, e.g., R_A and R_B parameters, e.g., as (R_A+R_B) (modulo K) where K is a system parameter that may indicate the maximum number of additional iterations.

In a seventh example, exchanged parameters (e.g., R_A, R_B) may also be used together with a pre-shared salt parameter associated to the connection to be established.

Alternatively, the exchanged parameters, e.g., R_A and R_B, may also be used together with (e.g., concatenated with) the shared password.

As a further alternative, the PAKE identifiers exchanged by device A and B in the preamble messages may also be used as input parameters in the above examples.

The above alternatives can be applied, e.g., when the PAKE is a balanced PAKE. They are also applicable, e.g., when the password in an augmented PAKE is stored in a secure element, e.g., a SIM card, that performs the PBKDF, and only returns a value or token from which the password cannot be easily retrieved, e.g., only through an offline dictionary attack. This value may be the verification value pair L and w0. The above alternatives are also applicable when a communication device requests a third communication device to compute and return such a value or token given the input exchanged parameters and a password that is not accessible or known to the communication device.

As a further alternative, the values R_A and R_B may also be combined at a later stage of the SEP to link the preamble to the key derived from the PAKE. For instance, once initiator and responder have established a shared secret K, a subsequent session key K′ may be derived from K, R_A and/or R_B.

It should be understood that deriving the salt parameter from the values exchanged in the preamble (such as the R_A and/or R_B), as described above, may be combined with limiting the rate of preamble messages or number of peer sessions, as described previously.

As indicated above, the PBKDF parameters, e.g., salt and/or counter value, may be received in a pre-configuration phase or may be exchanged through a different channel, e. g, they may be read by device A from a QR code (or bar code or other scannable code) attached to device B or may be exchanged out-of-band, e.g., through near field communication (NFC), BLE, or other wireless communication channels. These parameters may also be received during a pre-configuration from a central entity, e.g., a network function in a 5G system.

However, the exchange of the PBKDF parameters may require additional space, e.g., in the QR code. For instance, a QR code may need to encode more information and thus, it may need to be larger taking extra space on a package or on a product such as a light bulb. Moreover, a wireless message may need to be longer requiring additional energy for its transmission.

A way to deal with this issue is that the PBKDF parameters are implicitly exchanged, i.e., the actual information for the PBKDF parameters may be read or exchanged for a different purpose, e.g., to identify a given network or device type, and this information may be reused as the PBKDF parameters (e.g., salt or counter value). This can also be beneficial since it may allow the initiator to skip the exchange of the preamble messages, i.e., the initiator may directly send the first PAKE message.

FIG. 6 schematically shows a block diagram of an implicit PBKDF parameter generator element or function according to an embodiment.

For instance, if a field is exchanged/read for a first purpose (1^{st} PP) 64, then the same bits or a (predetermined) subset of the bits may be reused as salt parameter or counter value. To achieve this, an extractor function (EXTR) 62 (e.g., a register or addressable memory portion) may be used for copying the bits and supplying them to a PBKDF parameter memory or register (PDKDF-P) 66. Examples of the first purpose may include identification of the network or details concerning the devices such as their type. In particular, the first purpose may be anything other than the PBKDF operation.

The bits of the exchanged/read field(s) may also be expanded, e.g., by concatenating (a subset of) them to a predefined bitstring or by using them as input to a function, e. g, a hash function.

Implicitly signalled PBKDF parameters are advantageous in that QR codes can remain small and the PAKE process is linked to information contained in the QR code and used for other purposes.

The implicit exchange of the PBKDF parameters may limit the control over their values, and thus at least some PBKDF parameters, e.g., counter value, may be set to a non-suitable value. This can be prevented by adding a fixed value (e.g., number) as an offset to the implicitly derived value, so that the parameter value to be used (e.g., count value) may be calculated by the following function:

parameter value=fixed number+value obtained implicitly

or any other function that guarantees a certain property, e.g., a minimum value of the PBKDF parameters.

It is to be noted that a password may also be pre-configured, in particular, in embedded devices without an input device such as a keyboard. An example of such an embedded device is a light bulb. The password should not be exchanged unprotected over the air, but its reading may also be implicit. For instance, device B may have a QR code encoding certain parameters. Device A may then read the QR code and derive the shared password from all or at least a part of the read parameters. It is to be noted that the password may also be received during a pre-configuration from a central entity, e.g., a network function in a 5G system.

FIG. 7 shows a flow diagram of a conditional PBKDF parameter exchange according to an embodiment, which may be implemented by the PAKE control unit or function 46.

The preamble message Pre_AB may be configured to provide an indication whether device A knows already the required PBKDF parameters or not. Reason for this may be that in some use cases device A may have other access to the PBKDF parameters (e.g., via implicit signaling as described above).

However, in other scenarios it may not be feasible for device A to know the PBKDF parameters, e.g., when device A is a commissioning tool sending a request to many devices over WiFi or Ethernet or Bluetooth or the like. Similarly, in other scenarios device A may always know the PBKDF parameters.

Similarly, in some scenarios, device A may be a commissioning tool and device B may be an IoT device. Since IoT devices have very different communication and computational capabilities, a given IoT device B may only support a certain security level. Thus, device B may be configured or requested to indicate, e.g., in the preamble Pre_BA transmitted from device B to device A, its security strength that determines kLen (e.g., as configured by the manufacturer) and possibly the strength of a subsequent PAKE. Upon reception of Pre_BA, device A may derive suitable parameters to execute the PAKE using suitable security parameters.

An attacker may use this flexibility (e.g., introduced by a protocol giving an indication whether device A knows PBKDF parameters or not) to disrupt the communication in multiple ways. For instance, the attacker may desire to intercept the parameter information to better predict the potential output of the PBKDF. The attacker can trigger this exchange by placing itself between devices A and B and modifying a respective indication in the Pre_AB message requiring the exchange of the PBKDF parameters. In another example, an attacker may desire to modify the parameter information to easily force a failure in the subsequent PAKE phase. An attacker can do this by placing itself between devices A and B and modifying the exchanged information.

In the present embodiment, security and robustness of the protocol can be increased by providing a conditional transmission of, e.g., the PBKDF parameters.

In step 710, device B checks if it is aware about the knowledge of, e.g., its PBKDF parameters by device A.

Note that in some cases, a third device (e.g., a managing entity such as a network function in the 5G core network or an application running in the cloud) may perform such a check on behalf of device B. In such a case, device B may (need to) forward (or be requested to forward) the request to the third device, The third device would then perform the check protocol transcript and provide device B with the result of the check.

If device B (or a third device on behalf of device B) determines in step 710 that device A cannot know, e.g., its PBKDF parameters, the procedure branches to step 720 and device B sends, e.g., its PBKDF parameters, e.g. in preamble message Pre_BA, even if the content of message Pre_AB indicates that device A does have (access to) the PBKDF parameters (since message Pre_AB may have been maliciously modified).

Otherwise, if device B determines in step 710 that it is aware that device A knows, e.g., its PBKDF parameters, the procedure branches to step 730 and device B does not send, e.g., its PBKDF parameters, in message Pre_BA even if the content of message Pre_AB indicates that device A does not have (access to) the PKBDF parameters.

It should be understood that the PKBDF parameters of device B may have been determined by methods described previously such as deriving a salt from the preamble parameters (e.g., R_A and/or R_B) or by an OOB method. The conditional sending of the PKBDF paraments then may act as a further security enhancement, on top of the other measures.

In an example, device B may be configured to report or log an event where a preamble message with values different than expected gives an indication to device B of an ongoing attack.

Due to the advent of quantum computers, new cryptographic primitives (algorithms) for key encapsulation and digital signatures may be required. Once a quantum computer is available, many conventional cryptographic primitives will be broken, including, e.g., SPAKE2+.

Thus, an ecosystem, e.g., a smart home ecosystem, that starts deploying a system running a protocol based on PBKDF plus SPAKE2+ or another SEP may need a protocol upgrade to incorporate a quantum resistant PAKE. For instance, a different PAKE protocol (e.g., as described in Oleg Taraskin et al.: “Towards Isogeny-Based Password-Authenticated Key Establishment” or Xinwei Gao et al.: “Efficient Implementation of Password-Based Authenticated Key Exchange from RLWE and Post-Quantum TLS”) may be executed in the PAKE phase after the initial preamble phase.

While a device, e.g., device A, may be capable of running multiple PAKE protocols, another device, e.g., device B, may only be capable of executing one PAKE protocol (either the legacy SPAKE2+ or a new PAKE protocol, e.g., a quantum-resistant PAKE protocol). In this example, legacy devices may only be able to run SPAKE2+ while new devices may run a new PAKE protocol.

FIG. 8 shows a signaling diagram of a preamble exchange with PAKE support information according to an embodiment.

In this embodiment, a modified preamble message Pre_BA 802 may contain a new protocol support field (PAKE-SUPP) indicating the PAKE protocol(s) supported by device B. In an example, the protocol support field may contain a SPAKE2 ID. If device A receives and reads the corresponding field, then device A knows which PAKE protocol it needs to execute. If device A does not receive the new protocol support field, then device A knows that device B is a legacy device executing e.g. SPAKE2+.

Similarly, a modified message Pre_AB 804 may include the new protocol support field (PAKE-SUPP) indicating the PAKE protocol(s) supported by device A. If a new device B receives the protocol support field, then device B knows that device A supports multiple PAKE protocol(s) and may pick up a suitable one.

Similarly, a modified message Pre_AB 804 may include a request to receive the new protocol support field from device B.

It should be noted that other signaling parameters or information described in other embodiments may be combined with the information in the new protocol support field. Moreover, further information such as how the key derivation parameters (e.g., salt and count values) were/are to be obtained or that the other party used an OOB method.

Furthermore, in the future, certain parameters of the PBKDF and/or PAKE protocol may be customizable, e.g., the hash function used in the PBKDF. However, this requires that both device A and B know which algorithm to use.

To address this problem, information about preferred parameters of device A, e.g., the preferred hash function or the supported hash function, may be exchanged in message Pre_AB during the preamble phase.

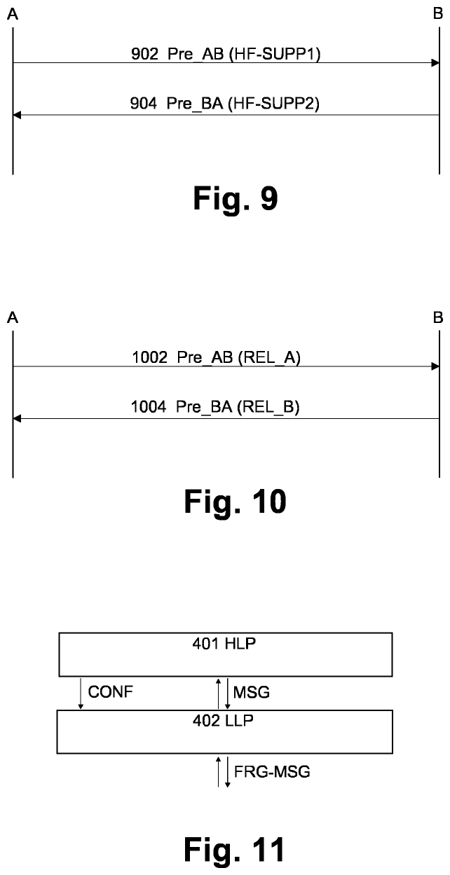

FIG. 9 shows a signaling diagram of a preamble exchange with hash function information according to an embodiment.

To address the above problem, device B may make a choice about the used hash function upon reception of a modified Pre_AB message 902 with a new hash function support field (HF-SUPP) and provide (signal) its choice in a modified Pre_BA message 904 with the new hash function support field (HF-SUPP).

Legacy devices unable to understand the new hash function support field of the embodiment may just ignore this field. If no corresponding answer is returned, then device A may be configured to use legacy parameters.

In some cases, message exchange in the preamble and/or PAKE phase may occur over an unreliable transport layer, i.e., messages may be dropped and, e.g., retransmissions may be required. This may require an enhancement of the protocol itself with capabilities for retransmission of messages or an exchange of certain reliability parameters, e.g., as part of the Pre_AB and/or Pre_BA messages, with a lower layer protocol (e.g., MAC protocol) that may provide such reliability capability.

The reliability capability may involve at least one of denoting a message as reliable so that upon reception of a message, the receiver needs to send an acknowledgement back; including a field that denotes a message to act as an acknowledgment for a given previous message (e.g., identified by means of a message identifier such as a counter); and including a maximum number of retransmissions required for a message (e.g., until an acknowledgment is received).

FIG. 10 shows a signaling diagram of a preamble exchange with reliability information according to an embodiment.

According to the embodiment, reliability settings REL_A and REL_B (e.g., reliability settings 402 in FIG. 4) are exchanged in the preamble phase, e.g., in modified Pre_AB message 1002 and a modified Pre_BA message 1004 with corresponding new fields and may apply to all subsequent messages in the protocol or to specific (e.g., marked or predetermined) messages that require reliability.

An underlying reliability protocol may allow for a limited number of pending acknowledgments and retransmissions, e.g., a single one. Device A and device B, e.g., when executing the preamble and PAKE protocol; may be configured to enforce that they do not accept or allow receiving a given message before a previous one has been properly accepted or received and an answer has been provided.

For instance, if device A sends a message M_A1, then device B replies with a message M_B1, then device A replies with a message M_A2, then device B replies with a message M_B2, then M_A3, then M_B3 and so on. Then, one of the devices, e.g., device A, may be configured not to accept message M_Bi before message M_A(i-1) has been acknowledged.

In a first example, upon reception of the reliability parameters REL_A and REL_B in the Pre_AB and Pre_BA messages, devices A and B may be configured to apply reliability parameters that ensure the highest reliability from the two sets of reliability parameters. For instance, if reliability parameter REL_A requires up to two retransmissions and reliability parameter REL_B requires up to three retransmissions, then both devices A and B are configured to retransmit up to three times.

In a second example, upon reception of the reliability parameters REL_A and REL_B in the Pre_AB and Pre_BA messages, devices A and B may be configured to apply reliability parameters that ensure the lowest reliability from the two sets of reliability parameters. For instance, if reliability parameter REL_A requires up to two retransmissions and reliability parameter REL_B requires up to three retransmissions, then both devices A and B are configured to retransmit up to two times.

In a third example, upon reception of the reliability parameters REL_A and REL_B, devices A and B may be configured to apply the reliability parameters required by the other party. For instance, if reliability parameter REL_A requires up to two retransmissions and reliability parameter REL_B requires up to three retransmissions, then devices B and A may retransmit up to two and three times, respectively.

The reliability parameters REL_A and REL_B may be preconfigured as a set of configurations, e.g., four possible configurations (can be binarily encoded with two bits) that correspond to different parameters. In an example, the preamble field may only include an identifier identifying the chosen configuration.

In above example, the possible configurations may correspond to configurations with increasing reliability. Device A and B may then be configured to select e.g. a configuration with highest/lowest offered reliability based on a pre-deployed and/or configurable policy.

Certain networks may have an MTU of limited size. While SPAKE2+ has small message length, the usage of a future PAKE protocols may involve longer messages. Thus, it is a challenge to determine how to fragment messages and transmit them in a reliable manner.

FIG. 11 schematically shows a block diagram of a lower layer protocol setting for fragmentation handling according to an embodiment.

Thus, in an embodiment, the reliability parameters exchanged in an initial configuration phase (e.g., exchanged in the Pre_AB and Pre_BA messages pf the preamble phase of SPAKE2+) may include an MTU parameter (e.g., MTU parameter 407 of FIG. 4). The exchange MTU parameter(s) may refer to the MTU of the physical layer of device A, of device B, or of devices A and B. This MTU parameter(s) together with the PAKE version (e.g., PAKE version 404 in FIG. 4) provide an indication about fragmentation requirements, e.g., fragment size and/or number of fragments of subsequent messages.

In an embodiment, the devices A and B may not be aware of the allowed MTU (e.g., this may have been pre-configured). Therefore, the messages exchanged in an initial configuration phase (e.g., preamble phase in SPAKE2+, SPAKE2, a PAKE, or another SEP) may include a parameter such as the size of the subsequent messages in the protocol so that fragmentation requirements in the subsequent protocol messages can be derived/determined, e.g., whether certain messages in the subsequent protocol will require fragmentation and how many fragments.

These parameters may be used to set a lower layer protocol handling fragmentation.

As indicated in FIG. 11, a lower layer protocol (LLP) 402 may be configured to handle both fragmentation and reliability.

The configuration (CONF) of a lower layer fragmentation protocol may be implicit since the lower layer protocol 402 can monitor the length of a message (MSG) passed/forwarded by a higher layer protocol (HLP) 401 and determine based thereon the number of required fragments of fragmented messages (FRG-MSG) and based on this number the specific reliability parameters. This configuration can also be explicit, e.g., by passing/forwarding the maximum size of subsequent messages, e.g., PAKE messages, in an initial message, e.g., the preamble messages of the PAKE or another SEP.

The above parameters (e.g., MTU size and/or message size) may be used in the protocol shown in FIG. 3 to fragment certain messages, in particular, in an embodiment, messages PAKE1, PAKE2, PAKE3, and PAKE4 may be fragmented, where messages PAKE1 and PAKE2 may have the highest chances of requiring fragmentation.

In an example, fragments may include a fragment identifier.

In another example, the configuration message may provide an indication to the lower layer fragmentation protocol whether subsequent messages sent by, e.g., device A, may be fragmented and transmitted together. For instance, device A may require the transmission of messages 1 and 2 of length 1200 bytes each while the MTU is 1000 bytes. If fragments cannot be transmitted together, then a total of four fragmented messages need to be transmitted (e.g., 1000 bytes, 200 bytes, 1000 bytes, and 1000 bytes). If fragments can be combined, then only three fragmented messages need to be transmitted (e.g., 1000 bytes, 1000 bytes, and 400 bytes).

In an embodiment, a receiving party may be configured not to reply before all fragments of a certain message, e.g., PAKE 1, have been received and reassembled.

The underlying reliability/fragmentation protocol executed at a device (e.g., device A) may require requesting from another device (e.g., device B) the specific fragments (of a given PAKE message) that have not been received, e.g., received before a timer expires. Alternatively, a device may also confirm the fragments that have been received.

An entity in the device (e.g., the PAKE protocol (PAKE control unit or function 46 of FIG. 4) itself or an underlying reliability/fragmentation protocol on behalf of the higher layer protocol 401) may be configured not to send a subsequent (PAKE) message before all fragments of the previously expected (PAKE) message have been received.

The reliability parameters applied by a lower layer reliability/fragmentation protocol may depend on the fact whether a given message requires fragmentation or not. Furthermore, the reliability parameters may depend on the number of fragments.

In an example, if a message does not require fragmentation, then a retransmission may be required within a given timeout. If a message requires fragmentation of a message into multiple fragments, then a higher timeout value may be applied.

In another example, the lower layer reliability/fragmentation protocol may be configurable as to how many fragments of a message can be sent simultaneously.

In an embodiment, a party may request a specific fragment of a message if it has not been received before a timeout expires. For instance, if message PAKEI needs to be fragmented into two fragments, then device B may be configured to request the second fragment from device A if it has not been delivered before a given timeout.

This fragmentation capability (and such a lower layer reliability and fragmentation protocol) may be applied not only to a PAKE protocol, but also to similar cryptographic protocols or involving large messages or to other applications requiring fragmentation (e.g., because of the usage of quantum-resistant primitives). This may involve e.g., digital signatures or key encapsulation mechanisms.

In an alternative embodiment, an SEP including a PAKE may be configured to handle fragmentation of certain messages, e.g., by a lower layer protocol offering fragmentation/reliability capabilities to upper layers, e.g., the SEP.

As explained in connection with FIGS. 2 and 3, upon reception of PAKE1 and PAKE2 messages both the first device (e.g., device A) and the second device (e.g., device B) may be configured to derive secret keys that can be used for the protection of the subsequent communication.

In a specific example, SPAKE2+ or another SEP may define that a protocol transcript TT (e.g., protocol transcript 406 of FIG. 4 or as defined in Taubert, T. et al.: “SPAKE2+, an Augmented PAKE (Draft)”) may be generated by means of a KDF (e.g., at the PBKDF control unit or function 44 of FIG. 4) based on input parameters of SPAKE2+ (or the other SEP) and PBKDF.

The encoded bitstring TT of the protocol transcript is used to derive keys Ka and Ke. The key Ka is used for the generation of the confirmation messages (c_B, c_A) exchange in the PAKE3 and PAKE4 messages. The key Ke is used for the protection of subsequent traffic.

However, the protocol transcript does not depend directly on the information exchanged in the preamble, so that the initial preamble exchange and the subsequent PAKE messages are not well linked. Moreover, the Ke key is a single key used for the protection of the traffic, but certain protocols, e.g., 3GPP protocols, require two keys, one for integrity and one for encryption.

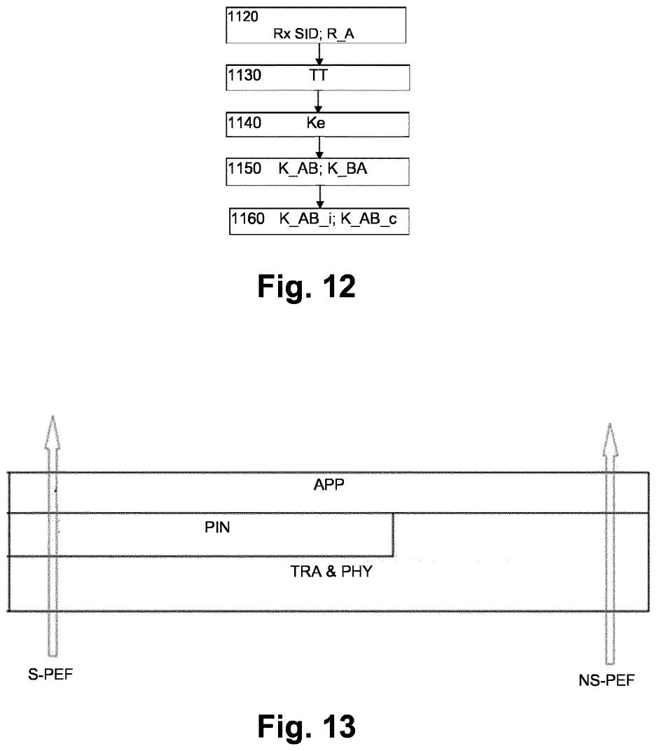

FIG. 12 shows a flow diagram of a key derivation procedure based on exchanged preamble information according to an embodiment.

In the embodiment, a KDF (e.g., PBKDF) is enabled to use input information exchanged in the preamble phase.

In step 1120 in the preamble phase, e.g., the session ID (SID) and/or the random values R_A and/or R_B are exchanged between devices A and B. This information is used in step 1130 to derive the protocol transcript (TT).

Then, in step 1140, the Ke key is derived from the protocol transcript.

In step 1150, the Ke key is used to derive by means of a KDF (e.g., PBKDF) two keys K_AB and K_BA to be used to protect the communication from the first device A to the second device B and vice versa.

Then, in step 1160, K_AB (or K_BA) is used to derive an integrity key and a confidentiality key K_AB_i and K_AB_c (or K_BA_i and K_BA_c) by means of a KDF (e.g., PBKDF) to ensure integrity and confidentiality of the messages sent from device A to device B (or from device B to device A).

In an embodiment, the integrity and confidentiality keys K_AB_i and K_AB_c and K_BA_i and K_BA_c may be directly derived from Ke by means of a single KDF call.

Such embodiments in which integrity and confidentiality keys are generated are, e.g., useful in the context of 3GPP standards where integrity keys are used to generate a message authentication code, e.g., by using a 5G New Radio Integrity Algorithm (NIA) and confidentiality keys are used to encrypt the messages, e.g., by using a 5G New Radio Encryption Algorithm (NEA).

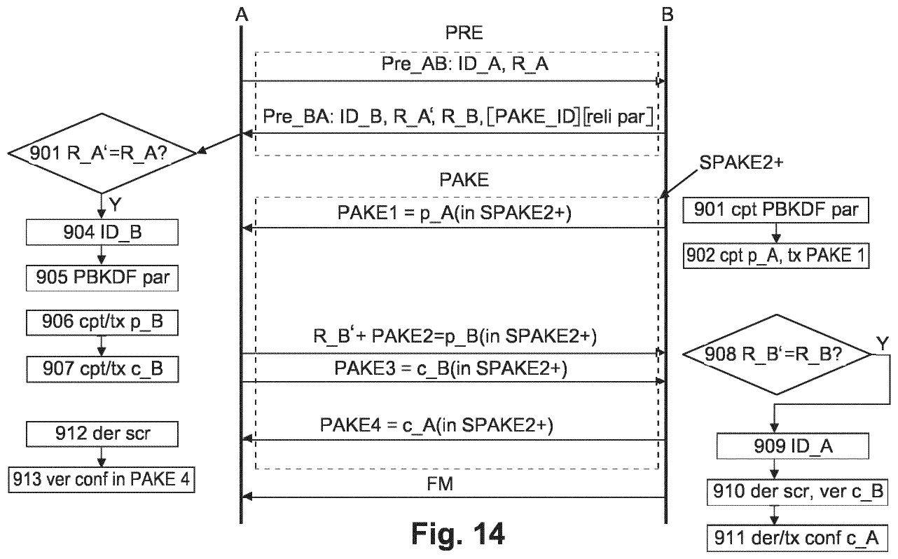

FIG. 14 schematically shows a signaling and processing diagram for an improved SPAKE process or other SEP with reduced number of roundtrips according to an embodiment.

The protocol design (preamble phase and PAKE phase) of FIG. 3 requires three round trips in total before the first device (e.g., device A) and the second device finish the exchange.

However, having a protocol involving multiple round trips leads to an increased latency of the protocol and a higher usage of, e.g., communication or energy resources. When a user is involved, e.g., a user using device A (e.g., a commissioning tool such as an app running on a phone) to configure device B, the user needs to wait for the protocol to be executed and a higher latency decreases user experience.

Thus, in the embodiment of FIG. 14, the messages of FIG. 3 are rearranged to require only two round trips in total, thereby decreasing protocol latency and thus increasing user experience. This is also useful in cases where the protocol is to be executed over a constrained network and/or with many devices simultaneously.

The way of achieving only two round trips, and thus, a more efficient protocol is to have the second device (e.g., device B) send the first PAKE1 message together with the Pre_BA message in step 902. This is feasible because upon reception of the Pre_AB message from the first device (e.g., device A), the second device may already execute in step 901 the PBKDF and generate the PAKE (e.g., SPAKE2+) parameters required for the first message PAKE1. Note that this may require rearranging some messages or operations in some PAKEs, e.g., in SPAKE2+ due to its unbalanced nature. For example, a second value could be sent in the first PAKE1 message and a first value could be sent in the second PAKE2 message. In the case of the process described in T. Taubert et. al: “SPAKE2+, an Augmented PAKE (draft-bar-cfrg-spake 2plus-03)”, 6 Jul. 2021, section 9, the value Y would be sent in the first PAKE1 message and the value X would be sent in the second PAKE2 message where the values X and Y are defined as in the SPAKE2+ paper. Note that in some cases it may also require the second communication device to request a user or a third communication device the retrieval of the password or a password-based value, e.g. the verification value pair L and w0 in SPAKE2+ in case the second communication device has not been previously provided or configured with it.

It is to be noted that in such a protocol design device B may be subject to an increased risk of denial-of-service attacks by an attacker sending many initial Pre_AB messages, which may, e.g., exhaust the available PSIs. However, to avoid this problem, the present embodiment may be combined with the above limitation embodiment with the limitation settings based on the limitation parameters 405.

It is further to be noted that the first device (e.g., device A) only needs to process PAKE1 if the Pre_BA message succeeds or is acceptable, e.g., if the check (R_A′=R_A) in step 903 is affirmative and PBKDF is executed properly in step 905. This also holds if messages Pre_BA and PAKE1 are combined in a single message. If these verifications are confirmed, device A can proceed to process message PAKE1, compute its public share and transmit PAKE2 message in step 905, derive keys (e.g., as described in FIG. 12) for later communication and transmit PAKE3 in step 907. These messages PAKE2 and PAKE3 may optionally include R_B′.

Device B, upon reception of R_B!, PAKE2 and PAKE3 may check R_B′ for consistency in step 908, process PAKE2 and verify the correctness of PAKE3. If this holds, security keys (e.g., as described in FIG. 12) for the subsequent communication can be derived in step 910 and message PAKE4 can be computed and sent to device A in step 911 as acknowledgement that the protocol was successful. If R_B and R_B′ do not match, device B may abort the process, e.g., with an error message or a report (e.g., to identify man-in-the-middle attacks or other attacks) or drop the executed protocol or restart the protocol.

Finally, in step 912, device A derives the shared symmetric secret from the PAKE4 message and verifies the confirmation in step 913.

In contrast to the embodiment of FIG. 3 where the finish message (FM) is sent by the second device (e.g., device B) to the first device (e.g., device A) so that the first device is aware that the second device has (successfully) finished the exchange, the present embodiment of FIG. 14 does not require this finish message since the first device deduces the state of the second device from the reception of the fourth message PAKE4. If some fields in the finish message are required by device A, then those fields may be sent together with the PAKE4 message.

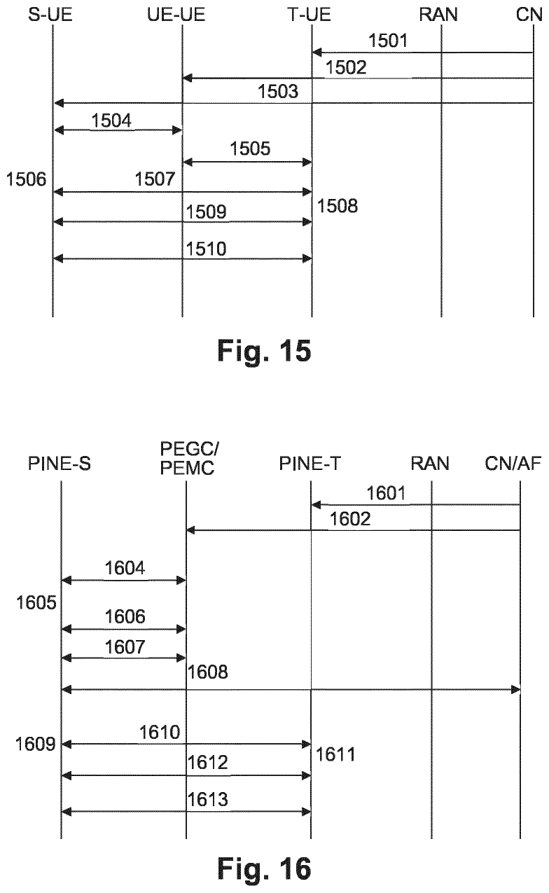

FIG. 15 schematically shows a signaling and processing diagram for UE-to-UE relay scenarios according to an embodiment. In FIG. 15, a Source-UE (S-UE) and a target UE (T-UE) may want to establish a secure communication over a UE-to-UE UE relay (UE-UE) making use of the PC5 interface. For instance, the Source-UE may be a smart health sensor, the target UE may be a smart watch, and the UE-to-UE relay may be a mobile phone. The three UEs may be connected to a core network (CN) over a radio access network (RAN). The devices may also be out-of-coverage at some point of time. To this end, the UEs may be initially configured in steps 1501, 1502, 1503. These configuration steps may involve a request by each of the UEs to the CN requiring authorization to make use of UE-to-UE relay services. This request may be initiated upon primary authentication of each of the UEs. The authorized UEs may be provided in steps 1501, 1502 and 1503 with respective security keying materials for a subsequent discovery phase and/or security keying material or authorization information to setup a secure communication channel. In steps 1504 and 1505, the Source-UE and the UE-to-UE relay as well as the target UE and the UE-to-UE relay may perform a discovery process, e.g., as described in TS 33.503. For instance, the UE-to-UE relay may be an announcing UE and the target and Source-UEs may be monitoring UEs. The UEs may also perform a model B discovery that involves two discovery messages. In steps 1506 and 1508, a password may be entered in the target and Source-UEs, e.g., in the case of a balanced PAKE. The password, or a value derived from it, may also be retrieved from the parameters configured in steps 1501 and 1503, e.g., in the case of an augmented PAKE. The Source-UE and the target UE may then establish a secure and authenticated channel. To this end, the Source-UE and target UE may run the protocol (procedure) of FIG. 3 or FIG. 14 over the UE-to-UE relay. In step 1507, the source and target UEs may exchange one or two messages that may correspond to the preamble messages described in above embodiments. These messages may include a session ID, a random value, an indication of PBKDF parameters, or any other parameters as described above. These messages may also include a relay service code or any identifier related to the type of communication service to be established. In step 1509, the source and target UEs may exchange PAKE messages to establish a secure authenticated channel. The source and target UEs may also optionally exchange additional messages for key confirmation. Finally, in step 1510, source and target UEs can securely communicate over the UE-to-UE relay.

It is to be noted that the preamble may be exchanged in the discovery phase for efficiency purposes.

It is to be noted that the discovery phase itself, with or without preamble, may also be based on a PAKE.

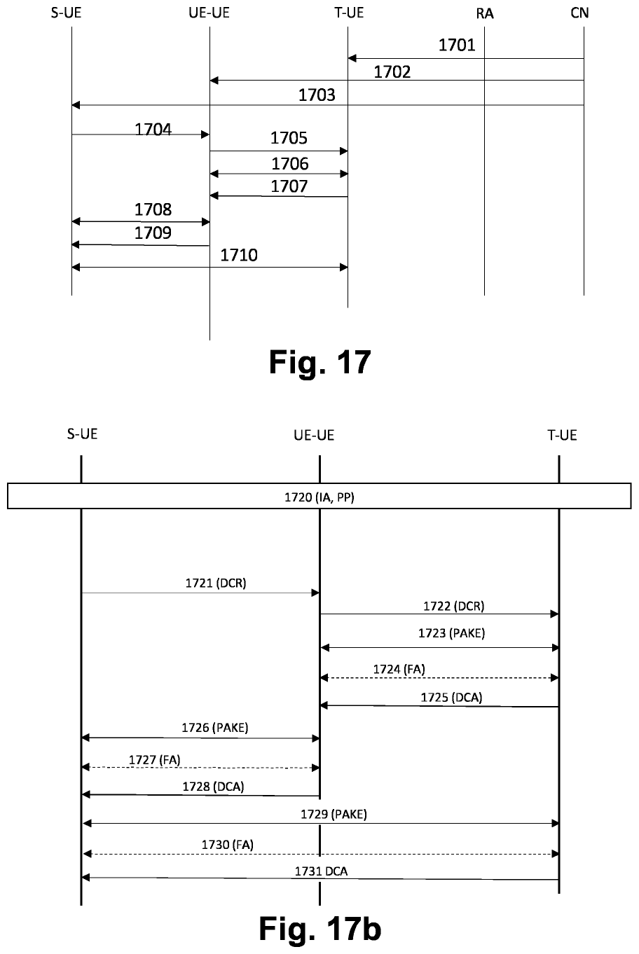

In an embodiment that can be combined with other embodiments, the security establishment between a Source-UE (S-UE)) or a target UE (T-UE)) and a UE-to-UE UE relay (UE-UE), e.g., as in the procedure introduced in FIG. 15, may also be done as indicated in above SEP embodiments. For instance, the security establishment between T-UE and UE-UE/S-UE and UE-UE may be integrated/performed in/after Steps 1504 and 1505, e.g., as shown in FIG. 17.

FIG. 17 schematically shows a signaling and processing diagram for UE-to-UE relay scenarios according to an embodiment. In FIG. 17, steps 1701, 1702, 1703 refer to an initial authorization and parameter provisioning. Step 1704 can refer, for example, to an initial discovery message or an initial Direct Communication Request from S-UE to UE-UE. Step 1705 can refer, e.g., to an initial discovery message or an initial Direct Communication Request from UE-UE to T-UE. In step 1706 one or multiple steps or messages described in the embodiments may be performed. It is to be noted that step 1705 may include fields in Pre_AB as described in above embodiments. Step 1707 may be, for example, a Direct Communication Accept message. This step may include fields in the last message in the embodiments, e.g., a last confirmation message or the PAKE4 message. In step 1708 one or multiple steps or messages described in the embodiments may be performed. Step 1709 may be a direct communication accept and it may also include fields in the last message in embodiments, e.g. a last confirmation message. In step 1710 one or multiple steps or messages described in the embodiments may be performed.

In a related embodiment that can be combined with other embodiments, the password used in the previous embodiment and described in FIG. 17 may be configured during the initial authorization and parameter provisioning phase or generated by a device and exchanged with another device over an out-of-band (OOB) channel, or maybe be entered by a user.

In a related embodiment that can be combined with other embodiments, the SEP used in the previous embodiment and described in FIG. 17 may rely on a balanced or an augmented PAKE.

In a related embodiment that can be combined with other embodiments, upon the execution of the PAKE-based SEP that involved devices, e.g., S-UE or UE-UE or T-UE, may exchange an access token that may include an identifier, the identifier of the associated session, the user identifier, the identifier of groups of the user, privileges, access rights, etc. This information in the token may have been signed by the CN or an application in the CN and provided to a UE during the initial authorization and provisioning phase (e.g., steps 1701, 1702, 1703 in FIG. 17). This token should be stored in a secure location, e.g., SIM card in the UE, upon delivery to a UE. This token should be exchanged over a secure channel, e.g., once the SEP has been executed.

In a related embodiment that can be combined with other embodiments, upon the execution of the PAKE-based SEP that involved devices, e.g., S-UE or UE-UE or T-UE may receive an access token that may include an identifier, the identifier of the associated session, the user identifier, the identifier of groups of the user, privileges, access rights, etc. This information in the token may have been signed by the CN or an application in the CN and provided to a UE during the initial authorization and provisioning phase (e.g., steps 1701, 1702, 1703 in FIG. 17). This token could be received over a secure channel, e.g., once the SEP (such as a PAKE-based SEP) has been executed. The UE receiving the token may have been configured with a policy in the initial authorization and provisioning phase (e.g., steps 1701, 1702, 1703 in FIG. 17) that allows determining whether the UE sending the token is authorized to utilize the UE-UE service. Such a communication flow is illustrated in FIG. 17b, similar to FIG. 17, where DCR refers to a Direct Communication Request, DCA refers to a Direct Communication Accept and steps 1724, 1727 and 1730 (Further Authorization(FA)) are based on such an authentication token. In step 1720, initial authorization (IA) and parameter provisioning (PP) are performed.

In a related embodiment that maybe combined with other embodiments, the PAKE1 message may be sent in the initial DCR message from S-UE to UE-UE and then to T-UE. The PAKE2 message may then send back in the DCA message from T-UE to UE-UE relay and then to S-UE. For instance, in steps 1721, 1722, 1725 and 1728 in FIG. 17 b. A device or method according to this embodiment may use an implicit key confirmation in which PAKE3 and PAKE4 messages are not included. A device according to this embodiment may transmit messages with fields related to Pre_AB in the DCR message to indicate such things as the password to use, parameters for key derivation, etc.

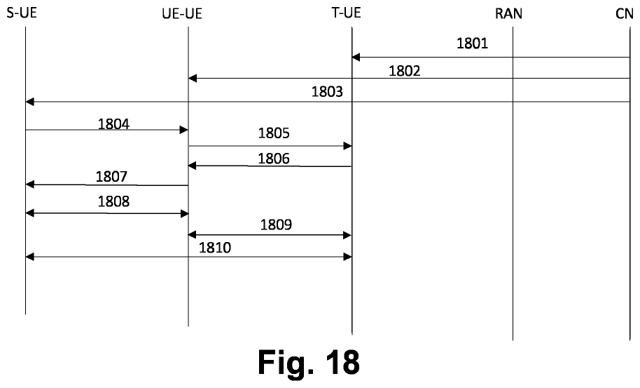

In a related embodiment illustrated by means of FIG. 18, steps 1801, 1802, 1803 refer to an initial authorization and parameter provisioning. Steps 1804 and 1805 refer to an initial discovery solicitation message. Steps 1806 and 1807 refer to a subsequent discovery response. Step 1808 refers to the establishment of a secure PC5 link between S-UE and UE-to-UE. Step 1809 refers to the establishment of a secure PC5 link between UE-to-UE and T-UE. Step 1810 refers to the establishment of a secure PC5 (L2) or communication (L3) link between S-UE and T-UE. The security aspects in steps 1808, 1809, and 1810 may be based on a PAKE-based SEP as described in the previous embodiment illustrated by means of FIG. 17 and optionally on a further authorization phase relying on authorization tokens.

In a related embodiment that can be combined with other embodiments, the access token contains data related to the password used in the SEP. This allows linking the PAKE-based SEP to the token exchanged at a later stage.

In a related embodiment that can be combined with other embodiments, if an augmented PAKE is used in the SEP, then the UE-UE relay may have been configured with password-based values (such as the verification value pair L and w0 in SPAKE2+) to ensure that it is difficult for a UE-UE relay to impersonate the S-UE or T-UE. Thus, step 1706 of FIG. 17 may involve a Pre_AB message or PAKE1 message first sent by T-UE to the UE-UE device. Thus, step 1708 of FIG. 17 may involve a Pre_AB message first sent by the UE-UE device to the S-UE and to which the S-UE replies with a Pre_BA and/or PAKE 1 message.

In a related embodiment that can be combined with other embodiments, if an augmented PAKE is used in the SEP, then the S-UE and T-UE may have been configured with password-based values (such as the verification value pair L and w0 in SPAKE2+) to ensure that it is difficult for these devices to impersonate the UE-UE relay. Thus, step 1706 of FIG. 17 may involve a Pre_AB message first sent by T-UE to the UE-UE device and replied by a PAKEl message from the UE-UE relay to the T-UE device. Thus, step 1708 of FIG. 17 may involve a Pre_AB message or PAKE1 message first sent by the UE-UE device to the S-UE.

In a related embodiment that can be combined with other embodiments, if an augmented PAKE is used in the SEP, then the T-UE may have been configured with password-based values (such as the verification value pair L and w0 in SPAKE2+) to ensure that it is difficult for the T-UE to impersonate the S-UE. Thus, step 1710 of FIG. 17 may involve a Pre_AB message or PAKE1 message first sent by S-UE to the T-UE device.

In a related embodiment that can be combined with other embodiments, the balanced PAKE may be CPAKE and the augmented PAKE may be OPAQUE.

In a related embodiment that can be combined with other embodiments, the key confirmation messages in PAKE3 and PAKE4 may be done implicitly as in, e.g., CPAKE, removing the need for the exchange of PAKE3 and PAKE4.

In a related embodiment that can be combined with other embodiments, during the initial authorization and parameter provisioning phase, e.g., related to FIG. 18, the UE-to-UE relay or Source-UE or target UE may be configured with User Info ID, Relay Service Code(s), UE-to-UE Relay Layer indicator(s), Traffic type (IP or non-IP); the UE-to-UE Relay Layer Indicator indicates whether a particular RSC is offering 5G ProSe Layer-2 or Layer-3 UE-to-UE Relay service, default destination layer-2 IDs, security related parameters for discovery or the PAKE such as a password or a password-based value or further authorization process such as an authorization token or a policy or cryptographic key material to verify a token, validity time of the parameters.

In a related embodiment, provisioning of security parameters may be done by 5G Direct Discovery Name Management Function (DDNMF), Prose Key Management Function (PKMF), or the PCF.

In a related embodiment that can be combined with other embodiments, the discovery keys and/or passwords and/or public key and/or policies required are configured in the initial authorization and provisioning phase (e.g., steps 1701, 1702, 1703 in FIG. 17). These may be required for such things to discover other UEs and/or perform a (PAKE-based) SEP and/or to verify the access token and/or to verify the authorization of a UE based on the token and a policy. A PKMF might be in charge of such parameters such as a public-key. A UE might contact the DDNMF to obtain the PKMF address. In case multiple networks are involved and some of the devices are in roaming (e.g., the Source-UE), the PKMFs of the respective networks (home and serving) might need to share with each other some of those parameters, in particular, the public key used by each of them and linked to the secret private key. Once this sharing is done, any relevant public keys can be configured in the initial authorization and provisioning phase. If this is done, then the PKMF of a UE (e.g., Source-UE) might be in charge of signing the access token. Additionally or alternatively, when a UE (in roaming) performs the initial authorization and provisioning phase, a UE might first contact its PKMF and from its own PKMF might be routed to the PKMF in charge of the discovery and PC5 keying materials including the management of access tokens (i.e., the PKMF of the home network). This PKMF might be in charge of the management of the public key linked to the private key used to sign the access token. This PKMF might manage authorization policies or rely on the PCF. The PKMF might thus retrieve (locally or from the PCF) a policy for the UE, the PKMF might also assign access rights to the UE and place them in an access token that can subsequently sign. This signed access token is then configured in the UE.

In a related embodiment that can be combined with other embodiments related to parameter provisioning in and out of coverage, the provisioning of passwords can be done either in coverage or out-of-coverage in Step 0. When the provisioning is done when a UE is in coverage, the UE is configured with one or multiple passwords “raw-passwords” or “password-derived tokens”. Each “raw-password” or “password-derived token” is associated with “metadata”. The “metadata” may include “password hint”, “expiration date”, and “access rights”. The field “password hint” indicates which password to use; the field “expiration date” indicates how long a password is valid; the field “access rights” indicates which authorization provides a password. The bitstring used as “password” in a PAKE is computed as the KDF of the “raw-password” and the associated metadata fields. In the case of an augmented PAKE, the “password-derived token” may be derived from the “password” itself. As in other embodiments, the parameters can be received from the 5G PKMF and 5G DDNMF when the UE is in coverage.

In a related embodiment that can be combined with other embodiments or used independently related to parameter provisioning in-and out-of-coverage, during provisioning in-coverage, a UE is also provisioned with an “out-of-coverage policy” determining whether a UE may use a temporary password (or credentials, e.g., key) entered or provisioned by the user when the UE is (or was) out-of-coverage. When in out-of-coverage, a UE can be provisioned with a temporary password entered by the user if “out-of-coverage policy” allows for it. This “out-of-coverage policy”, which has been configured in the initial authorization and provisioning phase when the UE is in coverage, might also be used to control other communication or security aspects when the UEs are out-of-coverage including a) the security protocol that needs to/is allowed to be used or b) the security credentials to use. For instance, a UE might be capable of setting up a secure PC5 connection over a UE-to-UE relay when it is out-of-coverage, e.g., based on some of the above embodiments or based on some solutions, e.g., in TR 33.740. Note that this may only be allowed if the UE has been configured with a policy allowing it to do so. For instance, when a UE is configured when it is in coverage, (for example, following the configuration in above embodiments or in Solution #3 in TR 33.740 or in above embodiments), the UE can be configured with a policy regarding the type of protocol or configuration parameters to be used when out-of-coverage, e.g., parameters related to Solution #4 in TR 33.740. Once a UE, e.g., the Source-UE, has performed the discovery phase with a UE-to-UE relay, the UE can send a direct communication request message. If the said UE is out-of-coverage and the preconfigured policy allows for it, the said UE is allowed to use the out-of-coverage solution. In this case, the UE will send a Direct Security Request message as in Solution #4 in TR 33.740 or may request the user to enter a short password (e.g., if no password is available or passwords have expired). This DCR message may include a flag indicating the solution choice (e.g., due to out-of-coverage reasons), although this flag might be implicit to the transmission of out-of-coverage parameters. For instance, if the UE-to-UE relay receives a DCR message with a specific field, e.g., an authorization token as in Step 2 in Solution #4 in TR 33.740, then the UE-to-UE relay might proceed with Step 3 in Solution #4 in TR 33.740 instead of Step 3 in Solution 3 in TR 33.740. For instance, if the UE-to-UE relay receives a DCR message with an out-of-coverage flag, the UE-to-UE relay might request the user to enter an out-of-coverage password (if the policy allows it). Furthermore, upon reception of this DCR message, the UE-to-UE relay device may need to (based on a policy received when in-coverage) check whether the connectivity environment is out-of-coverage and the out-of-coverage solution is applicable. If this is not the case, the UE-2-UE relay may not reply to the DCR message, or alternatively, the UE-2-UE relay may request the UE a DCR message including the in-coverage parameters so that an in-coverage solution can be used for the establishment of the PC5 security.

In a related embodiment that can be combined with other embodiments or used independently related to parameter provisioning and solutions to use in-coverage and out-of coverage, it is advantageous if the verification of the type of solution and parameters to be used is performed before a key establishment and authentication phase. In particular, it is advantageous if the verification of the Source-UE authorization (Step 4 in Solution #4 in TR 33.740) is performed before the Direct Authentication and Key Establishment Procedure (Step 3 in Solution #4 in TR 33.740) where the Source-UE authorization is performed by verifying the authorization token received in the DCR message (Step 2in Solution #4 in TR 33.740). The advantage is that, in this case, the risk of Denial of Service is reduced. It is also advantageous in that it saves resources (Step 3) in a case where the UE is not authorized. In the case of Solution #4 in TR 33.740, the verification may involve the verification of the authorization token and/or the verification of the out-of-coverage environment. It should be noted that the above holds in the case of, e.g., Sol #4, because the verification of the Source-UE authorization in Sol #4 is based on authorization tokens that are shared in the DCR message, and the sharing needs to be done by default in a secure way as explained in other embodiments. Since the receiving party (e.g., UE to UE relay) already has the authorization token at that point of time (after receiving the DCR message), then the receiving party can check authorization without delay. This is not always possible. For examples, Step 1724 in FIG. 17b may not be executed before Step 1723 because the authorization token exchanged in Step 1724 is protected based on the secure link established in Step 1723.

Thus, in a further related embodiment, the exchange of the authorization token may be integrated into step 1723 itself. For example, the authorization token may be exchanged as soon as a key is established in step 1723. For instance, referring to FIG. 14, an authorization token may already be included in the message, PAKE 2. If a PAKE (or in general, a SEP) is integrated/implemented by means of Direct Auth and Key Establish Request and a Direct Auth and Key Establish Response messages (and/or Direct Security Mode Command and the Direct Security Mode Complete) as in TS 36.536, the authorization tokens may be exchanged in those messages as soon as the underlying SEP (transported by means of, e.g., the Direct Auth and Key Establish Request and a Direct Auth and Key Establish Response messages) allows protecting the authorization token. This may require defining a new message field: “Authorization Token” that allows indicating that one of those messages includes one or more protected authorization tokens.

In a related embodiment, the authorization token exchanged in Step 2 may be advantageously protected as follows. Instead of using the approach in TS 33.503 Clause 6.3.5.2 (that can only protect up to 256 bits of data) and where the selected key in Step 1 in TS 33.503 Clause 6.3.5.2 is used to generate a keystream (Step 2 in TS 33.503 Clause 6.3.5.2) that is used to encrypt the private fields of the message (Step 3 in TS 33.503 Clause 6.3.5.2), the selected key in Step 1 in TS 33.503 Clause 6.3.5.2 is used as (or, used to derive an) encryption key to be used in combination with a NR Encryption Algorithm (NEA) to encrypt the private fields of the message, e.g., the authorization token. An advantage of this approach is that the authorization token (that is longer than 256 bits) exchanged in Step 2 in Sol#4 in TR 33.704 can be protected. Another advantage is that this same technique can be used to protect the authorization token sent by the UE-to-UE relay to the Source-UE, e.g., in Step 5 in Sol#4 in TR 33.704 where the input fields to the NEA algorithm might differ, e.g., the DIRECTION field might be set to 0 in Step 2 and to 1 in Step 5.

In a related alternative embodiment, it may be advantageous in some cases if the authorization token is not exchanged in the DCR message (Step 2 in Solution #4 in TR 33.740) but just before verification of the authorization token (Step 4 in Solution #4 in TR 33.740) and Direct Authentication and Key Establishment procedure (Step 3 in Solution #4 in TR 33.740) as in other embodiments in this application. An advantage of this embodiment is that both authorization tokens associated to Source-UE and UE-to-UE relay can be protected in a similar way, namely by using the keys derived during the Direct Authentication and Key Establishment procedure. An additional advantage is that this embodiment does not require changes in TS 33.503 Clause 6.3.5 used to protect the DCR messages since current protection is limited to a maximum of 256 bits.

In a related embodiment that can be combined with other embodiments or used independently related to parameter provisioning in and out-of coverage, the negotiation about the type of protocol or parameters (in-coverage or out-of-coverage) to use may be negotiated or signalled or verified in the initial discovery phase.

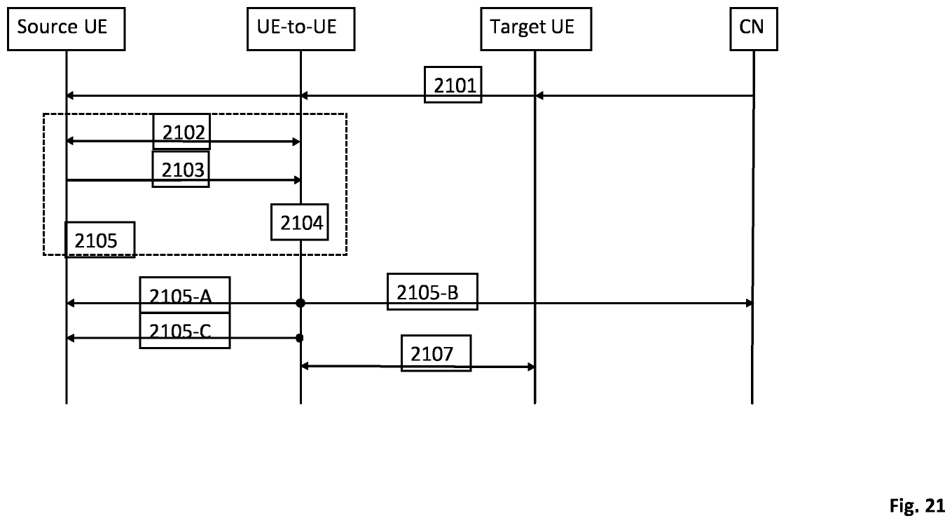

An embodiment is illustrated in FIG. 21 wherein in Step 2101, the UEs perform an initial authorization and parameter provisioning for in-coverage and out-of-coverage situations. This step includes the configuration of parameters for different solutions/SEPs for in-coverage and out-of-coverage security establishment and authorization. In Step 2102, the Source-UE and UE-to-UE relay perform a discovery phase. In Step 2103, the Source-UE determines its out-of-coverage (in-coverage) situation and chooses a security solution/SEP and/or security parameters for its out-of-coverage (in-coverage) situation. The Source-UE builds correspondingly a Direct Communication Request based on the chosen out-of-coverage (in-coverage) solution and security parameters. It may include a SEP identifier. In Step 2104, the UE-to-UE relay receives the DCR message and determines whether the DCR message either implicitly or explicitly contains parameters for an out-of-coverage (in-coverage) solution/SEP. The UE-to-UE relay then checks that the requested solution/SEP and security parameters fit its out-of-coverage (in-coverage) situation and whether a policy deployed in Step 2101 together with any authorization information received in Step 2103 allow the usage of said solution/SEP and security parameters. Once this policy is evaluated and if the result is positive, the UE-to-UE relay will proceed executing (or facilitating the execution of) the requested out-of-coverage (in-coverage) solution/SEP. Alternatively, if the result is negative, the UE-to-UE relay may-based on configuration-indicate the Source-UE policy mismatch, and potentially request a different DCR message including a requested solution/SEP and security parameters. In Step 2105, the UE-to-UE relay may take one of the following alternative steps:

Step 2105-A, send a message to the Source-UE to go on with an in-coverage solution. Step 2105-B, send a message to the core network to go on with an out-coverage solution. Step 2105-C, send a message to the Source-UE indicating that the security establishment based on the exchanged parameters is not feasible and requesting alternative parameters. It is to be noted that the above steps can be repeated for the security establishment process and authorization between Target UE and UE-to-UE relay. It is to be noted that the above process illustrates how UEs signal and agree on solution and security parameters to use by means of the DCR message.

This signaling and agreement phase might also be carried out in the discovery phase.

In a related embodiment, the UE-to-UE relay might have indicated in the discovery message to the Source-UE or target UE whether it (the UE-to-UE relay) is in coverage or out-of-coverage. This can be done in discovery model A if the UE-to-UE relay operates as the announcing UE. This can be done in discovery model B if the UE-to-UE sends a response discovery message to the Source-UE indicating whether it is in-coverage or out-of-coverage.

In a related embodiment, the relay service code indicates the type of service that is to be used by the 5G ProSe Source/Target and UE-to-UE relay, and thus, the RSC implicitly indicates the type of (security) solution/procedure (e.g., in coverage or out-of-coverage, i.e., with support of the core network or not) that is preferred. For instance,
a Source-UE might use multiple (e.g., two) different types of relay service code to discover UE-to-UE relays capable of offering the establishment of a secure communication link with and without network assistance.
For instance, the service code may indicate the type of security procedure that is required and/or preferred.

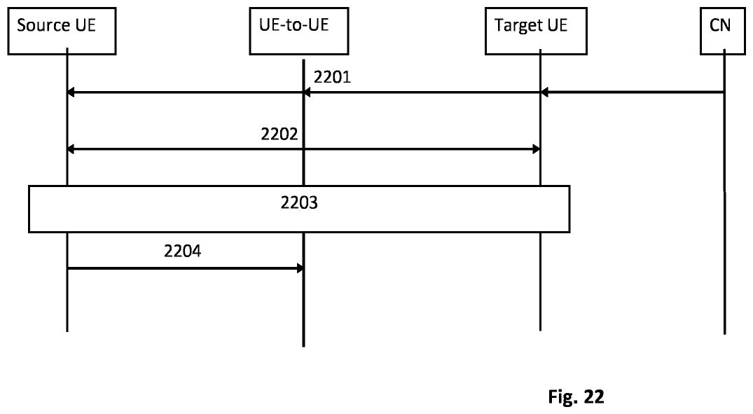

A related embodiment wherein the signaling and agreement is performed in the discovery phase is illustrated in FIG. 22. In FIG. 22, Step 2202 refers to the initial configuration and authorization step in which Source-UE/target UE/UE-to-UE relay devices are configured; step 2202 refers to the initial discovery that might be model A or model B; step 2203 is the policy evaluation in which at least one of Source-UE, target UE, UE-to-UE relay devices might determine the preferred solution (e.g., in-coverage or out-of-coverage solution. i.e., security procedure with network support or without network support) to use; step 2204 refers to an initial message used to trigger the setup of the security setup of the UE-to-UE relay security.

In a variant that might be combined with other variants, in Step 2202, the Source-UE might send a discovery solicitation message (discovery model B) that may be forwarded by the UE-to-UE relay to the target UE. The target UE may reply with a response discovery message to the Source-UE over the UE-to-UE relay. In another variant, in Step 2202 the Source-UE might send an announcement message (discovery model A) that is the received and forwarded by the UE-to-UE relay to the target UE. In another variant, in Step 2022 the UE-to-UE relay is the device sending the announcement/solicitation messages to Source-UE and target UE, and potentially, receiving response messages from the Source-UE and target UEs. In these solicitation and announcement discovery messages, the Source-UE and/or UE-to-UE relay and/or target UE might indicate their in-coverage or out-of-coverage situation as well as preferences for the type of in-coverage/out-of-coverage solution (i.e., security procedure with network support or without network support). In the response discovery messages, the UEs might indicate their preferences and/or choices.

In a variant that might be combined with other variants, in Step 2203 one or more of the devices may evaluate a configured policy and may decide which solution(s) to use, are preferred to use, or might be proposed to use. For instance, the target UE might have been configured with a policy that allows selecting a preferred solution that is then proposed (e.g., by means of a message) to UE-to-UE relay and Source-UE. For instance, the target UE might be configured to indicate (by means of a message) one, two or more solutions in preferred usage order. For instance, upon reception of the message sent by the target UE, the Source-UE might verify the preferred choice(s) of the target UE and evaluate it against the evaluation of its own policy, e.g., that might give priority to either its own preference or the preference of the other party. For instance, any of the Source-UE, UE-to-UE and target UE may evaluate the policy and choose a preferred solution. Then, an exchange of proposal and acceptance/confirmation may occur between the Source-UE, UE-to-UE and target UE.

In a variant that might be combined with other variants, Step 2202 and Step 2203 might happen concurrently, e.g., Step 2203 happens at the target UE upon reception of a solicitation discovery message and before sending response discovery message.

In a variant that might be combined with other variants, Step 2203 happens at the Source-UE upon reception of the response discovery message.

In a variant that might be combined with other variants, the UE-to-UE relay choice, e.g., as done during discovery, might depend on the evaluation of the policy specifying the preferred out-of-coverage/in-coverage solution. For instance, an out-of-coverage UE-to-UE relay might deprioritized compared with an in-coverage UE-to-UE relay. For instance, an in-coverage UE-to-UE relay allowing an out-of-coverage (e.g., because of recent/non-expired security parameters) might be prioritized compared with an in-coverage UE-to-UE relay with outdated out-of-coverage parameters.

In a variant that might be combined with other variants, a UE (e.g., UE-to-UE relay) receiving message in Step 2204 may still disagree on the choice made by the sending UE (e.g., Source-UE) and reply in a similar way as in above embodiments and/or as described in FIG. 21.

In a variant, a UE might require UE-to-UE communication due to emergency reasons. In this case, the UE, e.g., a Source-UE, may include an emergency indication such as an “emergency RSC” when distributing, e.g., discovery messages or sending an initial DCR message. The UE-to-UE relay receiving this indication may determine how to process the message based on, e.g., its context (e.g., coverage situation) and/or a policy/configuration. For instance, the source/target UEs and/or UE-to-UE relay may, depending on context and policy, perform one or more of the following actions:
Request/provide emergency services based on the policy;
Log emergency requests based on the policy;
Handle an emergency request as a UE-to-UE communication and/or as a UE-to-Network communication depending on the context. For instance, if the UE is out of coverage, the communication is handled as UE-to-UE communication (i.e., communication is directed to other UEs in the area) and if in coverage, it is handled as UE-to-Network communication (i.e., communication is directed to the network) and/or UE-to-UE communication (i.e., communication is directed to other UEs in the area).
Provide/Request UE's security capabilities and/or PC5 signaling security policy.
Send an indication to the core network about the (provided) emergency service based on the policy;
Disable security or not enforce security even if the policy requires security under normal circumstances. This may apply to:
   The discovery messages exchanged between Source-UE and target UE over the UE-to-UE relay.
   The establishment of security in the PC5 links.
Allow/request the usage of temporary (e.g., entered by the users) credentials (e.g., passwords) to provide a minimum level of security based on the policy.

This approach has the advantage that communication over UE-to-UE relay may be used in emergency or safety-critical situations, e.g., to enable that two UEs that usually might not be able to interact with each other, actually interact with each other. For instance, the Source-UE might be the UE of person in need of help (e.g., because they are being attacked or because they are suffering an asthma event) so that when said person requests help, the help/emergency message may be distributed over a UE-to-UE relay to other UEs that are close by, and in this way, they may obtain help fast. For instance, the Source-UE might be a tag attached to a luggage and when the tag misses the connection to its UE (e.g., the UE of the owner of the luggage), the tag (acting as a Source-UE) might send an emergency message to try to reach “its” UE.

In a related variant, the message sent by the Source-UE may include the cause of the emergency or information related to the UE/person requesting help (e.g., a description of the UE/person or description of the luggage).

In a related variant, the message sent by the Source-UE may include the cause of the emergency or information related to the desired target UE/person (e.g., a description of the UE/person).

In general, the above embodiments require a policy to choose a preferred solution, in particular, a preferred in-Coverage and/or Out-of-Coverage solution (i.e., a security procedure with network support or without network support). This policy might be deployed to the source-UE and/or UE-to-UE relay and/or target UE. This policy may include one or more of the following:
Which of the supported solutions is preferred or to be used or authorized when in-coverage or when out-of-coverage by one or more of the involved devices, namely Source-UE, target UE, and UE-to-UE relay;
Which authorization mechanism is preferred or used when in-coverage or when out-of-coverage by one or more of the involved devices, namely Source-UE, target UE, and UE-to-UE relay;
Priorities assigned to different devices used to determine which solution is preferred, e.g., (1) in case that different devices have different preferences, and/or (2) priority might depend on the coverage status of each of the devices. For instance, if Source-UE is in-coverage and other UEs are out of coverage, then the Source-UE's solution is prioritized, and if all UEs are out of coverage, a default prioritization list determines which UEs solution is to be used;
Which parameters of a supported solution are preferred or are to be used or authorized when in-coverage or when out-of-coverage;
Whether a communication may/is allowed to be established in, e.g., an emergency situation (e.g., for emergency services), and which security credentials/security configuration/security mechanism are required e.g., whether null ciphering and/or integrity algorithms are allowed/tolerated;
Whether/how security parameters (such as a password, or a key, etc) of an out-of-coverage solution may be generated and/or entered when devices are out-of-coverage;
Which (default) authorization capabilities or access rights are assigned to the generated and/or entered (temporary) credentials (e.g., passwords, keys), when other credentials (e.g., long term credentials) have expired and the UEs (e.g., Source-UE and UE-to-UE relay) are out-of-coverage.
Which UE-to-UE relay is preferred in case multiple UE-to-UE relays are available may depend on their conditions (e.g., in coverage/out-of-coverage) and/or the conditions (e.g., in coverage / out-of-coverage) of the Source-UE (and/or target UE). For instance, an in-coverage UE-to-UE relay might be preferred in certain situations while an out-of-coverage UE-to-UE relay might be preferred in other circumstances;
Which security method/procedure is to be used or preferred for path switching or relay re-selection and based on which factors (e.g., coverage situation, performance requirements, etc), e.g., whether to use a standard security establishment procedure (e.g., a procedure that is used when no communication is available between Source-UE and target UE over a UE-to-UE relay, e.g., Sol #3, or #4, or #10 in TR 33.740), or skip it and use an optimized procedure, e.g., by re-using the already established security context (e.g., security policy and security algorithms) from the previous PC5 unicast link to (re-)establish the secure communication link over a new relay (e.g., as in Sol #14 in TR 33.740) e.g., for optimization purposes. For instance, if Source-UE and target UE have a secure communication link over a first UE-to-UE relay established with a security procedure without network assistance (because the first UE-to-UE relay was out-of-coverage), then an optimized security procedure (e.g., Sol #14 in TR 33.740) might not be preferred when performing path switch and moving to a second UE-to-UE relay (that is in coverage and can offer a security procedure with network assistance).
Whether it is allowed to have more than one active connection between a Source-UE and a Target-UE over multiple UE-to-UE relays, based on e.g., UEs coverage situations, and/or performance requirements, and/or resource constraints.
Priorities for the choice of a solution in a case of a duplicated security association using the same or different (types of) solutions. A first example of this may be where a Source-UE might start a first security association (e.g., with a DCR message) using an in-coverage (or out-of-coverage) solution and then (e.g., if a timer times out) it also sends a second security association (e.g., with a second DCR message) using an out-of-coverage (or in-coverage) solution, and at that point of time, the Source-UE might receive the reply for the first security association. The policy might specify (by indicating a priority) which solution is to be used in such a case. The priority in the policy might be applied, e.g., by the UE-to-UE relay or the Source-UE. A second example of this may be where a Source-UE might start a first security association (e.g., with a DCR message) using an in-coverage (or out-of-coverage) solution with a first UE-to-UE relay and send a second security association (e.g., with a second DCR message) using an in-coverage (or out-of-coverage) solution with a second UE-to-UE relay, and at that point of time, the Source-UE might receive the reply for the first security association. The priority in the policy might be applied, e.g., by the Source-UE or the CN;
Whether two UEs, e.g., Source-UE and target UE (that share/have a first secure communication link over a first UE-to-UE relay established with a security procedure without network assistance because when the first secure communication link was established the relay was out-of-coverage) should reestablish the security communication using a security procedure with network assistance if the first UE-to-UE relay is in-coverage again and/or a second UE-to-UE relay in-coverage is discovered.
A threshold/limit on the number of attempts that may be carried out when attempting to agree on a solution before, e.g., stopping the process and/or adding the other device to a blacklist and/or reporting the issue to the core network.

The End-UEs (i.e., Source and Target-UE) and the UE-to-UE Relay may have different preferences for which solutions and/or security parameters (e.g., password, cryptographic keys) to be used and thus may exchange negotiation messages e.g., as in FIG. 21 steps 2103, 2104, 2105, to either agree on a solution and/or security parameters or drop the connection. Negotiation relies on the security policies provisioned at the UEs and may take into account the coverage status, at the time of negotiation, of the UEs (e.g., End-UE and UE-to-UE Relay). The following embodiments may apply.

In an embodiment that may be combined with other embodiments or used independently, the End-UE may be in-coverage, while the UE-to-UE Relay may be out-of-coverage; the End-UE may request an in-coverage solution, i.e., a security procedure which requires network assistance, in which case, the UE-to-UE relay may approve it, if it is allowed by its security policy (it may also be requested, but the request may be rejected if the UE is not authorized to use that solution). In this case, it could be the End-UE that communicates (or facilitates the communication) and is assisted by the network in security establishment and authorization. For instance, TR 33.740 describes solutions (e.g., Sol #3) in which if the UE-to-UE relay is in coverage, the UE-to-UE relay is the entity that is relying on the network assistance to setup the secure link between UE-to-UE relay and target UE. For instance, Step 3. in Sol #3 in TR 33.740 v 0.6.0 is a Key Request including parameters received from one of the End-UEs, in this case, the target UE. If the UE-to-UE relay is out-of-coverage, but the target UE is in coverage, the target UE may act as a UE-to-Network relay for the UE-to-UE relay so that the messages of Step 3 are forwarded through the target UE (e. g, acting as a UE-to-Network relay). Similarly, the End-UE that is in coverage may also forward message in Step 4, Key response from the CN to the UE-to-UE relay.

In another related embodiment that may also be used independently, an End-UE (e.g., Source-UE) may:
Get indication from the UE-to-UE relay, e.g., including its identity.
send the contents of the initial DCR message to the network in, e.g., Step 3 in Sol #3, in a key request to the CN on behalf of the UE-to-UE relay,
receive the key response, e.g., Step 4 in Sol #3, from the CN on behalf of the UE-to-UE relay,
Send a DCR message including the contents of the received key response from the CN to the UE-to-UE relay.

This embodiment allows an End-UE (e.g., Source-UE) that may wish to use a UE-to-UE relay to directly use network assistance for establishing the security of that connection between UE and UE-to-UE relay. This embodiment has the advantage that reduces the number of exchanges messages.

This embodiment can also be applied in a similar way to the exchanges between a second End-UE (e.g., target UE) and UE-to-UE relay, for instance, the key request and key response that the (first) End-UE. UE exchanges may also relate to the keying material required for the establishment the secure link between UE-to-UE relay and the second End-UE (e.g., target UE).

In another embodiment, a first UE (e.g., a UE-to-UE relay) may be configured with a policy that determines whether it may perform a given type of security procedure (e.g., with network assistance) with the help/support of a second UE (e.g., an End-UE) when the first UE is out-of-coverage.

In another embodiment, the End-UEs may be out-of-coverage, whilst the UE-to-UE Relay may be in-coverage; the End-UEs may request an out-of-coverage solution, i.e., a security procedure which does not require network assistance, in which case, the UE-to-UE relay may approve of such solution if it is allowed by its security policy. Even if the UE-to-UE relay engages in a security procedure without network assistance, the UE-to-UE relay may still inform the network of the link establishment attempt between Source-UE and Target-UE for the network to provide its feedback (e.g., final authorization) on whether the communication is authorized or not (e.g., when Long Term Credentials have expired). Based on the network's feedback, the UE-to-UE relay may carry on relaying the messages or drop the communication if the network indicates that the connection is not authorized (e.g., if one of the End-UEs is not authorized or if it is opting for an out-of-coverage solution while being in-coverage).

In another related procedure, the UE-to-UE relay may request/allow the execution of a security procedure without network assistance, e.g., due to performance reasons or when out-of-coverage, between an End-UE (e.g., Source-UE or Target UE) and UE-to-UE relay. However, this communication link established with a security procedure without network assistance may be subject to a communication policy bound to a security procedure without network assistance (e.g., characterized by a given (limited) data rate, specific frequency range, (limited) temporal validity (e.g., up to t seconds), (limited) local validity).

In another related procedure, the communication policy may include an access control policy whose contents may be similar to the authorization policy in a PIN system as described in related procedures/embodiment variants.

In another related procedure, the communication link established with a security procedure without network assistance may be subject to a communication policy bound to a security procedure without network assistance.

In another related procedure, a security procedure of 5G ProSe PC5 Communication for 5G ProSe Layer-3 UE-to-UE Relay without network assistance may be as described in Clause 6.6.3.2 in TS 33.503 (draftCR in TDoc S3-233374).

In another related procedure, a security procedure of 5G ProSe PC5 Communication for 5G ProSe Layer-3 UE-to-UE Relay with network assistance may be as described in Clause 6.6.3.1 in TS 33.503 (draftCR in TDoc S3-233374).

In another related procedure, the UEs (e.g., UE-to-UE relay) may require (based on policy/configuration) the execution of a security procedure with network assistance or (re-)establishment of a secure link with network assistance between an End-UE (e.g., Source-UE or Target UE) and UE-to-UE relay if initially the End-UE (e.g., Source-UE or Target UE) and UE-to-UE relay established a secure link without network assistance, e.g., if the UE-to-UE relay was initially out-of-coverage or for performance reasons. When this new secure link is/needs to be established may depend on context (e.g., timeout, UE-to-UE relay again in coverage) or policy.

In a related procedure, the UE-to-UE Relay may be provisioned/configured with a policy/security parameter to support the execution of security procedures without network assistance when out-of-coverage, however, once in coverage, the UE-to-UE Relay may be required to provide a list of the (active) communication links between End-UEs for the network to check (e.g., whether UEs are authorized to communicate with each other, whether the security context (e.g., keying materials) need to be refreshed). The network may instruct the UE-to-UE Relay accordingly how to handle the communication links it established while out-of-coverage. For instance, it may drop the communication link between two End-UEs (e.g., due to timeout, or an authorization change), trigger a re-keying procedure, trigger a new security procedure, or maintain the communication link as is.

In a related procedure that may be combined with other procedures (such as the one described in the previous paragraph), the UE-to-UE relay may trigger the execution of a security procedure with network assistance by sending a message (e.g., security procedure request) whereby the message may be protected with the keying material of the current sidelink/PC5 communication link. Upon reception of the message, the End-UE (e.g., Source-UE) may provide the UE-to-UE relay with its credentials to perform a security procedure with network assistance, e.g., a Direct Communication Request including a Relay Service Code and PrukID as described in security procedure of 5G ProSe PC5 Communication for 5G ProSe Layer-3 UE-to-UE Relay with network assistance may be as described in Clause 6.6.3.1 in TS 33.503 (draftCR in TDoc S3-233374).

In another embodiment, one or more network functions (NFs) in the CN is in charge of monitoring whether security procedures based on a security procedure without network assistance are still allowed (e.g., as foreseen by the policies/security keying materials/credentials) provisioned to the UEs, or whether those policies/security keying materials/credentials have expired/been revoked, etc. This NF is then in charge of logging and/or stopping the security procedure currently in progress. This embodiment has the advantage of ensuring that the network has close control over which security connections may be or may not be established while UEs (End-UEs/UE-to-UE relay) can use security procedures without network assistance that may deliver enhanced performance (e.g., lower latency).

In an embodiment, a UE-to-UE Relay may send a message to an End-UE (e.g., step 2105-A in FIG. 21) to confirm the solution choice e.g., when there is a mismatch between the solution requested by an End-UE and the solution preferred by the UE-to-UE Relay, as the requested solution may be, upon policy verification, supported by UE-to-UE Relay. For instance, if Source-UE is in-coverage and requests an in-coverage solution, while the UE-to-UE Relay is out-of-coverage, the UE-to-UE relay may send a message to agree/go on with the in-coverage solution if its policy allows it and Source-UE could be assisted by the network. Similarly, if an out-of-coverage Source-UE requests an out-of-coverage solution, while UE-to-UE Relay is in-coverage, the UE-to-UE Relay will either send a message (e.g., step 2105-C in FIG. 21) indicating the mismatch or a message (e.g., step 2105-A in FIG. 21) informing the Source-UE of its approval of the Out-of-coverage solution. Similar indications may also be sent during the discovery procedure.

In an embodiment variant, in cases where the solutions (i.e., requested by an End-UE and preferred by UE-TO-UE Relay) match, the message sent in e.g., step 2105-A in FIG. 2 is not required.

In another alternative embodiment, or variant, it may be understood implicitly whether the UE-to-UE Relay supports the requested solution and approves it despite a mismatch, by simply continuing the procedure of link establishment based on the requested solution; in this case, Step 4-A may not be needed.

A UE-to-UE Relay may be provisioned with a policy which prohibits the establishment of communication links with End-UEs without network assistance regardless of whether it is in-coverage or out-of-coverage. If, for instance, a UE-to-UE relay attempts to serve and/or establish communication links with End-UEs without network assistance (e.g., an End-UE requests an out-of-coverage solution), the network may not be able to intervene (e.g., confirm to End-UE whether the UE-to-UE Relay is authorized to establish links when out-of-coverage), as UEs may be out-of-coverage. For such and other similar scenarios, where the network cannot assist or guarantee that communicating parties are rightfully authorized, UEs (e.g., Ends-UEs and/or UE-to-UE Relay) need to be able to verify whether the other party is authorized to use a solution that it requests/approves of, and embodiments to guarantee that may include, but are not limited to, the following.

In an embodiment, a service code, e.g., the Relay Service Code (RSC), which is provisioned by the network, may be associated with either an in-coverage/out-of-coverage solution, such that when it is used, the UE receiving the request or approval of a solution is assured that the requesting/approving UE is indeed authorized for an out-of-coverage solution.

In a further embodiment, if at least one of the UEs (e.g., Source, Target, or UE-to-UE Relay) is in-coverage, that UE may send a request, including the identifiers of the UEs and choice of security solution, to the network which then verifies and provides a final authorization for the links to be established.

In a further embodiment, UEs may be provisioned with different credentials (e.g., service codes, discovery keying materials, passwords, cryptographic keys, etc) which are associated with in-coverage and/or out-of-coverage solutions, such that the use of these identifiers/keys may implicitly imply that the UEs intend/are authorized to use the security solution requested or approved.

In a further embodiment, a UE, e.g., an End-UE, may construct a discovery message M that is protected with the keying materials associated with a given service code (e.g., relay service code). The UE may receive these keying materials/service code when the UE is authorized to use a given security procedure (e.g., in-coverage/out-of-coverage). The UE may choose these keying materials/service code if the application requires a given type of security procedure, e.g., with or without network assistance. This indicates the preferences of the UE. For example, a UE (e.g., an End-UE) may send a discovery message to another UE (another End-UE) over a UE-to-UE relay. The application specific fields of a given ProSe service may be protected with keying materials bounded to a service code such that the UE-to-UE relay or other UEs (that do not belong to or are associated to said application, e.g., ProSe service) cannot access them. A ProSe service may prefer/require the usage of a given security procedure, e.g., with or without network assistance. Based on the preferences, a UE might be eligible to receive credentials (e.g., a relay service code/keying materials) to select a UE-to-UE relay capable of providing the support for that given security procedure. These credentials are provided by the core network. The UE may then use these credentials to also protect or add protection to the discovery message M before sending it. This allows an application requiring a given type of security procedure to enable an End-UE to select UE-to-UE relays capable of providing the required type of security procedure for said application.

In a further embodiment, if an End-UE indicates a given preference for a given type of solution, e.g., in-coverage or out-of-coverage, i.e., with or without network assistance, and the UE-to-UE relay can provide support for such a solution/security procedure, the UE-to-UE relay does not need to indicate its coverage status since it is not required/useful for the End-UEs and this is advantageous to save bandwidth.

In a further embodiment, the End-UEs indicate their coverage status, e.g., in the discovery message, so that even if a UE-to-UE relay is out-of-coverage, the UE-to-UE relay may accept a request to use an in-coverage solution, i.e., a security procedure with network assistance, even if the UE-to-UE relay itself is out-of-coverage.

In a specific procedure based on the proposed embodiments, 5G ProSe End-UEs (e.g., Source and Target-UE) and a 5G ProSe UE-to-UE Relay may negotiate and select a security mechanism with or without network assistance based on a security policy. The negotiation procedure involves the following steps:
Step 0: Initial authorization of the UEs and provisioning/configuration with security parameters for different security mechanisms and policy for the negotiation/selection of a security mechanism.
Step 1: Source-UE and UE-to-UE Relay perform the discovery phase and indicate their coverage status.
Step 2: Source-UE builds a Direct Communication Request message including its chosen security mechanism (e.g., with/without network assistance) and security parameters based on local policy depending on e.g., its coverage situation, service preferences, UE-to-UE Relay coverage indication, etc.
Step3: UE-to-UE Relay receives Source-UE's DCR message and determines whether the DCR message contains an explicit security mechanism indication and/or parameters which implicitly indicate a preferred security mechanism (e.g., with or without network assistance). The UE-to-UE Relay verifies whether the security mechanism and/or security parameters fit its coverage situation and whether the policy provisioned in step 0, in addition to any authorization information received in Step 2 allow the usage of Source-UE's preferred security mechanism and security parameters. Based on the outcome of the evaluation, UE-to-UE Relay may either proceed with executing the requested security mechanism (e.g., with or without network assistance) or indicate a policy mismatch to Source-UE and request a different DCR message with its (i.e., UE-to-UE Relay's) preferred security mechanism and parameters.

The security mechanism negotiation is based on the policies provisioned to UEs in Step 0, which include, but are not limited to, the following:
Which of the supported security mechanisms or security parameters are preferred to establish a secure link with and without network assistance by the ProSe service.
Number of attempts permitted to agree on a security mechanism.
Default prioritization list that determines which UE's (e.g., Source-UE, Target-UE, and UE-to-UE Relay) security mechanism is prioritized when UEs prefer different solutions. The prioritization list may depend for instance on the UE's function (e.g., Relay) or its coverage status (e.g., in-coverage vs out-of-coverage).

In a particular procedure, the selection methods between security mechanisms with or without network assistance are as follows: a Network Assistance Security Indicator per Relay Service Code (RSC) is provisioned in the UEs to indicate whether to use the network assistance security procedure. The 5G ProSe End-UEs and the 5G ProSe UE-to-UE Relay select between security procedures with network assistance and security procedures without network assistance based on the Network Assistance Security Indicator. If an RSC associated with the enabled Network Assistance Security Indicator is included in Discovery message, then the PC5 security procedures with network assistance is performed between the End-UEs and the UE-to-UE Relay. Otherwise, if an RSC associated with the disabled Network Assistance Security Indicator is included in Discovery message, then the PC5 security procedures without network assistance is performed between the End-UEs and the UE-to-UE Relay. For 5G ProSe UE-to-UE Relay Communication with model A discovery, when the UE-to-UE Relay is in 3GPP coverage, the UE-to-UE Relay shall include the RSC associated with the enabled Network Assistance Security Indicator in Discovery Announcement message, then the End-UEs and the UE-to-UE Relay perform the PC5 security procedures with network assistance after discovery procedure. When the UE-to-UE Relay is out of 3GPP coverage, the UE-to-UE Relay shall include the RSC associated with the disabled Network Assistance Security Indicator in Discovery Announcement message, then the End-UEs and the UE-to-UE Relay perform the PC5 security procedures without network assistance after discovery procedure.

For 5G ProSe UE-to-UE Relay Communication with model B discovery, if the source End-UE includes in Discovery Solicitation message the RSC associated with the enabled Network Assistance Security Indicator, and the UE-to-UE Relay is in 3GPP coverage, then the End-UEs and the UE-to-UE Relay perform the PC5 security procedures with network assistance after discovery procedure. Otherwise, if the source End-UE includes in Discovery Solicitation message the RSC associated with the disabled Network Assistance Security Indicator, then the End-UEs and the UE-to-UE Relay perform the PC5 security procedures without network assistance after discovery procedure.

This procedure does not allow End-UEs that do not support PC5 security procedures with network assistance to discover each other in discovery model A when close to a UE-to-UE Relay that is in coverage. This procedure does not describe how source End-UE decides to include an RSC with or without Network Assistance Security Indicator in discovery model B. This procedure does not describe how a Source-UE can decide to perform a PC5 security procedure with or without network assistance if the Source-UE completes two discovery procedures, one with an RSC associated with the enabled Network Assistance Security Indicator and one with an RSC associated with the disabled Network Assistance Security Indicator.

To address these problems, multiple procedure variants that may be combined with each other or used independently may be applied:

In a procedure variant, the UE-to-UE relay in model A may send discovery messages with RSC associated with the enabled Network Assistance Security Indicator and discovery messages with RSC associated with the disabled Network Assistance Security Indicator when the UE-to-UE relay is in coverage. These messages with different RSCs may be sent alternatively or in any sequence or timing according to configuration and/or implementation.

In a procedure variant, the coverage status of the UE-to-UE relay (e.g., in Model A) is sent explicitly next to an RSC so that End-UEs can reply in a subsequent discovery message (e.g., in Model A) with their preferred solution (with or without network assistance).

In a procedure variant, an RSC may be associated to both a disabled Network Assistance Security Indicator and an enabled Network Assistance Security Indicator is not associated with any Network Assistance Security Indicator so that a UE receiving the RSC knows that it can chose, based on its preference, either a PC5 security procedure with or without network assistance. This RSC may be sent, e.g., in discovery model A, when the UE-to-UE relay is in coverage and supports PC5 security procedure with or without network assistance. This RSC may be sent together with the coverage status of the sending device (e.g., UE-to-UE relay) so that the receiving device can select a suitable security procedure.

In a procedure variant, the End-UEs in model A may be configured with a policy determining the preferred RSC (associated with enabled/disabled Network Assistance Security Indication) to react/reply to the received RSCs in the discovery messages so that a single PC5 security procedure (with or without network assistance) is executed. For example, if an End-UE receives two RSCs, one associated with enabled Network Assistance Security Indication and one associated with disabled Network Assistance Security Indication, the End-UE may have a policy preferring a PC5 security procedure without network assistance and will only react/reply to the discovery message with the RSC associated with disabled Network Assistance Security Indication.

In a procedure variant, the End-UEs and/or UE-to-UE relay may be configured with a policy determining a time window during which the End-UEs and/or UE-to-UE relay consider the RSCs received in discovery messages, which may be associated with enabled, disabled, or none of/both Network Assistance Security Indicator(s). The End-UEs and/or UE-to-UE relay may react/reply to or drop the received discovery messages based on the End-UEs preferred solutions determined by their policies so that a single PC5 security procedure is executed. Discovery messages received from the same Relay after the time window times out or after End-UE has already agreed to a security solution may be dropped.

In a procedure variant, when the UE-to-UE relay is in-coverage (e.g., in Model A) and transmits discovery messages with RSCs which are associated with enabled/disabled Network Assistance Security Indicator or both/none, the End-UEs (e.g., Source-UE and Target-UE) may have different preferences for the security solutions. For example, Source-UE may prefer a Network-assisted security procedure while Target-UE prefers a security procedure that is not network assisted. As such, whether the UE-to-UE Relay may support different security solutions for the first-hop-by-hop link and the second hop-by-hop link may be determined by its policy. Furthermore, if a single solution is required for both the first and second hop-by-hop links, the choice of the security solution is determined by the UE-to-UE Relay policy e.g., based on UE-to-UE preference or on the preference of the End-UE that responds to the announcement message first.

In a procedure variant, the End-UE in model B may be configured with a policy determining the preferred RSC (associated with enabled/disabled Network Assistance Security Indication) to use.

In a procedure variant, the End-UE in model B may be configured with a policy determining the PC5 security procedure (with or without network assistance) to execute if the Source-UE completes two discovery procedures, one with an RSC associated with the enabled Network Assistance Security Indicator and one with an RSC associated with the disabled Network Assistance Security Indicator.

In a procedure variant, when the negotiation of both security procedures with and without network assistance succeeds, i.e., in two parallel discovery procedures RSC(s) associated with an enabled or disabled Network Assistance Security Indicator are matched, then the End UE and UE-to-UE Relay shall perform the PC5 security procedure with network assistance. This requires one of the End UEs, e.g., the source UE to send a Direct Communication Request (DCR) message including the RSC bound to the PC5 security procedure with network assistance. If the UE-to-UE relay matched both RSCs in the previous discovery procedures, the UE-to-UE relay may then receive the DCR message and process it (according to Section 6.3.5 in TS 33.503) with the security materials bound to both RSCs following a predetermined order. For instance, the UE-to-UE relay may use first the security materials linked to the RSC bound to the PC5 security procedure with network assistance to security process the integrity verification and decryption of the DCR message. If this first security processing/verification fails, the UE-to-UE relay may try to process the integrity verification and decryption of the DCR message with the security materials bound to the PC5 security procedure without network assistance.

In general, the embodiments requiring the configuration and usage of a policy to determine which solution/protocol to use and which security parameters (e.g, passwords, cryptographic keys) to be used has the advantage that it may help prevent that a UE capable of using an in-coverage solution and an out-of-coverage solution decide to use the out-of-coverage solution, when in-coverage. A UE so doing not only breaks interoperability, but it can also introduce security weaknesses if the out-of-coverage solution is more relaxed (security wise) than the in-coverage solution.

This signaling and agreement phases might also be carried out in integrated discovery, I.e., the discovery messages are integrated in the DCR message. For instance, Solution #32 in TR 33.740 v0.6.0 describes an integrated discovery procedure that allows the establishment of a communication link between Source-UE and Target UE when the UE-to-UE relay is in coverage, I.e., with support of the network. The initial DCR message sent in such a solution/procedure by the Source-UE might also indicate the preferred type of security to use for the subsequent security establishment, e.g., with or without relying on the core network. Source-UE/UE-to-UE relay/target UE will then use the preferred security establishment based on the indication, context, and pre-configured policies.

In general, there is described a method and apparatus, which can be implemented in a device, for:
receiving an indication on the SEP (solution/protocol) and/or security parameters to use from a first device, said SEP (solution/protocol) and/or security parameters being to establish a secure link between a first and a second device,
evaluating whether the indicated SEP (solution/protocol) and/or security parameters can be used, and
processing said positive or negative evaluation (that is, sending a subsequent message determined by a positive or negative evaluation of the received indication) to the first or a third device based on the policy evaluation.

In general, there is described a method and apparatus, which can be implemented in a device, for:
receiving an indication on the SEP (solution/protocol) and/or security parameters to use from a first device, said SEP (solution/protocol) and/or security parameters being to establish a secure link between a first and a second device,
evaluating whether the indicated SEP (solution/protocol) and/or security parameters can be used.

In general, it is described a method and apparatus as above wherein the indication of the SEP (solution/protocol) and/or security parameters to use is signaled in a discover message or in a DCR message.

In general, it is described a method and apparatus as above wherein the indication on the SEP (solution/protocol) and/or security parameters to use is signaled in a discover message or in a DCR message and is implicit in the usage of a specific service, e.g., (relay) service code.

In general, it is described a method and apparatus capable of processing said positive or negative evaluation (that is, sending a subsequent message determined by a positive or negative evaluation of the received indication) to the first or a third device based on the policy evaluation.

Being able to manage out-of-coverage provisioning offers flexibility. Given that UE's are mobile, it is probable that they will be out of coverage from time to time. Despite their being out of coverage, it may be convenient for provisioning to occur during this time. For example, where user action is required, it may be convenient for the user to complete their actions at a time when the UE happens to be out of coverage.

In a related embodiment (that can be combined with other embodiments or used independently) which is related to parameter provisioning and solutions to use in-coverage and out-of-coverage, it is advantageous if the verification of the type of solution and parameters to use is performed before a key establishment and authentication phase. In particular, it is advantageous if the verification of the Source-UE authorization (Step 4 in Solution #4 in TR 33.740) is performed before the Direct Authentication and Key Establishment Procedure (Step 3 in Solution #4 in TR 33.740). It is advantageous since in this case the risk of Denial of Service is reduced. It is advantageous since it saves resources (Step 3) in case the UE is not authorized. In the case of Solution #4 in TR 33.740, the verification may involve the verification of the authorization token received in the DCR message in Step 2 and/or the verification of the out-of-coverage environment.

In a related embodiment that can be combined with other embodiments or used independently related to parameter provisioning in-and out-of-coverage, the negotiation about the type of protocol or parameters (in-coverage or out-of-coverage) to use may be negotiated or signaled or verified in the initial discovery phase.

In general, requiring the configuration and usage of a policy to determine which solution/protocol to use and which security parameters (e. g, passwords) to use is advantageous since otherwise a UE capable of using an in-coverage solution and an out-of-coverage solution might decide to use the out-of-coverage solution, when in-coverage. This not only breaks interoperability, but it can also introduce security weaknesses if the out-of-coverage solution is more relaxed (security wise) than the in-coverage solution.

In a related embodiment that can be combined with other embodiments related to the parameters exchanged prior to the PAKE execution, several parameters need to be exchanged prior to the PAKE execution in Steps 3, 6, and 9. These parameters may include the Relay Service Code or a “password hint” used to identify the “raw password”. In the absence of an explicit password hint, then the RSC serves as password hint, i.e., the password is linked to the RSC. In particular and in reference to FIG. 17b, the DCR message in Step 1721 includes the RSC and password-hints for the PAKEs in Steps 1726 and 1729; the DCR message in Step 1722 includes the RSC and password-hints for the PAKEs in Steps 1723 and 1729. When the UE-to-UE relay receives message of Step 1721, the UE-to-UE relay may remove the password hint for the PAKE in Step 1726. Furthermore, the UE-to-UE relay adds its password hint for the PAKE to the DCR message in Step 1722. When the target UE receives message in Step 1722, the target UE starts the PAKE in Step 1723 based on the received password hint.

In a related embodiment that can be combined with other embodiments related to PAKE execution, this embodiment clarifies how a PAKE can be executed in the context of SPAKE2 and SPAKE2+ that involve the exchange of four messages. The first and second PAKE messages are exchanged in a Direct Auth and Key Establish Request and a Direct Auth and Key Establish Response messages as in TS 33.536, respectively. The third and fourth messages are exchanged in the Direct Security Mode Command and the Direct Security Mode Complete as in TS 33.536, respectively.

In a related embodiment, the term long-term credentials, as specified in Clause 5.3.3.1.2.1 in TS 33.536, may also refer to passwords as used in a PAKE.

In a related embodiment that can be combined with other embodiments related to Secure exchange of data, the secure exchange of data relies on a shared key Ks between two UEs result of the PAKE. This key Ks is K_shared as defined in SPAKE2 or TT as defined in SPAKE2+. For a L2 UE-to-UE relay, Ks used as shared secret to derive PC5 keys for user/control planes and encryption/integrity by means of a key derivation function according to TS 33.220. For a L3 UE-to-UE relay, Ks is used to derive a PSK hint and a PSK. PSK and PSK hint are used in IKE-PSK to secure the L3 communication by means of a key derivation function according to TS 33.220.

In general, it is described a method and apparatus, which can be implemented in a device, for performing a second PAKE-based SEP between a first and a third device through the relay device. It is further described a method and apparatus related to the previous method and apparatus, for performing a first PAKE-based SEP between a first and a relay device, and if successful, authorizing the execution of the second PAKE based SEP.

### PERSONAL IoT NETWORKS

FIG. 16 schematically shows a signaling and processing diagram for personal IoT network (PIN) scenarios according to an embodiment. In the scenario of FIG. 16, a PINE source device (PINE-S), a PEGC and/or PEMC, and a PINE target device (PINE-T) are involved. These devices may be part of a PIN. For instance, the PINE source device may be a virtual reality (VR) device (e.g., a headset) that simulates vision to end up with a 3D environment in which a user appears to be immersed while browsing through it or experiencing it, the target PINE device may be a smart TV, and the PEGC/PEMC may be a mobile phone (UE). The three devices may need connectivity/services from a core network (CN) over a radio access network (RAN). These three devices may further need means for secure communication between each other. To this end, the devices may be initially configured in steps 1601 and 1602 and 1603 (as described above in connection with steps 1501 to 1503). The PINE source or target devices may not be directly configured by the CN because it may not be 3GPP RAN capable; instead, they may be configured by an application function (AF) through the CN or by the CN if the device is a 3GPP RAN capable device. The configuration may relate to security keying materials (e.g., PAKE-related, PC5 discovery keying materials as per TS 33.503) or other embodiments described herein, or communication parameters (such as QoS, required communication resources, and max. latency).

In step 1604, the PINE source device and the PEGC and/or PEMC may setup an underlying communication channel, e.g., a Wi-Fi channel. Then, the PINE source device may be triggered to enter a password, e.g., by means of a keyboard or by scanning a QR code (step 1605). This password may be generated by the PEGC and/or PEMC and it may be displayed on the screen. The password may also be derived by means of a KDF from a master secret on the PEGC and/or PEMC, e.g., the master secret of the UE used in primary authentication. The source PINE device and PEGC/PEMC are then able to exchange a preamble/PAKE messages/SEP in steps 1606 and 1607 as in the above embodiments resulting in a secure and authenticated channel. The PEGC/PEMC can then allow the source PINE device to make use of the 5GC communication resources in step 1608. This step may involve an authentication procedure with an AAA server, e.g., in the Application Function (AF) that would notify the PEGC/PEMC about the result.

In an additional variant, in step 1604, the PINE source device and the PEGC and/or PEMC may setup an underlying communication channel, e.g., a Wi-Fi channel. Then, the PEGC/PEMC may be triggered to enter a password, e.g., by means of a keyboard or by scanning a QR code, e.g., attached to the PINE source device. This password may have been provided by the 5CN or a third party on the PINE. The source PINE device and PEGC/PEMC are then able to exchange a preamble/PAKE/SEP messages in steps 1606 and 1607 as in the above embodiments resulting in a secure and authenticated channel and resulting in initial access to the CN or an AF making use of the CN 1608. Additionally, and prior to this step 1608, upon successful establishment of a secure and authenticated channel in 1607, the PEGC/PEMC may securely receive further security information from the PINE source device, e.g., a digital certificate allowing the PEGC/PEMC to verify the identity of the PINE source device. This digital certificate may be verified by the PEGC/PEMC or be forwarded for verification by the CN or AF. In this first case, the PEGC/PEMC may give the PINE source device access to the CN or an AF making use of the CN. In this second case in which the CN or AF perform the verification, the CN or AF would send a confirmation message to the PEGC/PEMC to allow/disallow the traffic from the PINE source device. Access to this AF making use of the CN may happen through a network exposure function (NEF). It is to be noted that step 1608 may give access to the AF through some specific network resources, e.g., specific to the target AF. For instance, an object of the AF may be to create a personal IoT network in which all devices are reachable. Thus, a PINE source device that succeeds in steps 1606, 1607, and 1608 may be allocated connectivity resources (e.g., IP address, communication resources ensuring a certain reliability/latency) matching the needs of the AF). The resources allocated to a PINE upon successful authentication/authorization in step 1608 may depend on:
a policy configured based on the user subscription in step 1602 and/or
the communication requirements for the PINE that may be configured in the PEGC/PEMC in step 1608 and/or
the amount of available resources taking into account other PINE devices connected to the PEGC/PEMC.

It is to be noted that the initial configuration in steps 1601 and 1602 and 1603 may be done by an external AF through the CN or by the CN. This initial configuration may include the distribution of information related to the SEP/PAKE, e.g., in an augmented PAKE or information allowing the verification of a subsequent credential (e.g., a digital certificate) received from the PINE source device upon establishment of a secure channel (e.g., by means of a SEP or PAKE-based SEP).

This initial configuration may also include communication policy for a PINE or communication policy for the PEGC.

In step 1609, a password/credentials may be entered/provided/used in the PINE source device and in step 1610 preamble messages as in FIG. 3 or 14 and above embodiments (equivalent to discovery messages) may be exchanged over the PEGC and/or PEMC to another PINE target device. The PEGC/PEMC may only allow traffic originated from PINE that has been already authenticated/authorized in a previous step. In step 1611, the same password may be made available to this device. Based on the (preamble) messages in step 1610 and shared password, both source and target PINE devices can run a PAKE as in above embodiments in step 1612 resulting in a secure communication channel between the PINE devices in step 1613.

In FIG. 16, CN may include several network functions including, e.g., AMF, SMF, or UPF. The UPF may interact with a PIN related AF located in the CN or outside of the CN. Configuration parameters may be provided/configured, e.g., by the PCF, UDM, or UDR.

In an additional (sub-)embodiment, an AF, in or outside of the 5G system, may be in charge of managing the PIN. This AF and the PEGC/PEMC establish a secure channel via Authorisation and Key Management for Applications (AKMA) (see TS 33.535). Once the PEGC/PEMC is authenticated based on the AKMA derived keys, the AF may provision the PEGC/PEMC with PIN configuration parameters (e.g., in step 1602). Before the AKMA anchor Network Function (AaNF) is allowed to provide the AF with an AKMA-derived AF key, the AKMA NF check with the UDM/UDR, directly or indirectly through the AUSF, whether the user of the PEGC/PEMC is authorized to setup a PIN as well as, e.g., subscription data, subscription authentication, PDU session information, QoS requirements.

In an additional variant, a PINE device, e.g., PINE target as in FIG. 16, supports a cellular interface, and thus, it be feasible to configure said device over a cellular interface, e.g., Uu interface or the PC5 interface, e.g., in step 1601. The initial configuration includes configuration parameters to setup the PC5 interface, e.g., discovery security material as described in TS 33.503. This initial configuration also includes PIN configuration information. This information may be provided, e.g., by the AF once the PINE (e.g., PINE target) has established a secure connection with the 5G CN (e.g., upon primary authentication) by means of AKMA derived keys that may allow performing an authentication/authorization procedure between PINE and AF. This initial secure PIN configuration may be performed over the Uu or PC5 interfaces. This initial PIN configuration may be performed before the PINE device has tried to join a PIN. If the PIN configuration is successful, the PEGC/M are also instructed/configured to route communication between PINEs, e.g., steps 1610, 1612, and 1613 may need to be routed over the PEGC/PEMC where the communication link between PINE source and PEGC/PEMC may be based on a first communication protocol (e.g., WiFi) and the communication link between PINE target and PEGC/PEMC may be based on a second cellular communication protocol. This configuration may be pulled by the PEGC/PEMC or pushed by the PIN AF. Thus, the PEGC/PEMC may be configured by the 5GC with a configuration policy determining that certain PC 5 traffic (e.g., at MAC or PDU session or IP layers) is to be routed to a different networking interface, e.g., a WiFi or a BLE or an IEEE 802.15.4 networking interface.

In an additional variant, a PINE device, e.g., PINE target in FIG. 16, may support a cellular interface. Such a PINE device may use non-regulated spectrum, and thus, it may not always be required to rely on resource allocation from the RAN.

In an additional variant, a PINE device, e.g., PINE target in FIG. 16, may establish a secure channel with the PEGC/PEMC by using a SEP, e.g., a PAKE-based SEP at application layer where the PAKE-based SEP messages may be transported, e.g., in a metadata field of PC5 discovery messages, or in other PC5 message.

In an additional variant, step 1608 may involve a(n) (mutual) authentication procedure between PINE and AF/5GC.

In a related variant, the PIN AF may play the role or include functionalities of an AAA server.

In a related variant, the following may occur.

At stage 1, a UE that is a candidate to become the PEGC/PEMC of a PIN performs initial (primary) authentication with the core network, e.g., using a root key (e.g., K_AUSF in 5G) shared by the UE and authentication entity in the CN (e.g., AUSF in 5G);

At stage 2, if this initial (primary) authentication is successful, the UE may request communication with a certain PIN AF; or the CN (e.g., AUSF) may check the subscriber preferences, e.g., related to PIN management. In this case, the CN may authorize the UE, and UE and PIN AF may setup a secure communication, e.g., using a K_AF derived from K_AKMA as in TS 33.535;

At stage 3, the UE and PIN AF can then perform an authentication/authorization procedure based on K_AF or a key derived from it using a Ua* protocol. Additionally, or alternatively, the procedure may also be a different high-level application protocol using some credentials specific to the PIN;

At stage 4, e.g., if the authentication/authorization is successful in stage 3, then the AF may inform the CN (e.g., AUSF, or AUSF through the AaNF, or other NF) of the UE status and may provide some configuration parameters related to, e.g., the PIN communication requirements, membership, etc that the UE has to use in its role of PEGC/PEMC. The CN (e.g., AUSF) may store this information in a data base, e.g., in the UDM (UDR). The CN (e.g., AUSF) may check this received configuration against the user subscription and verify whether it fulfils it. Billing information may also be updated due to the new status of the UE (active PEGC/PEMC).

At stage 5, the CN (e.g., AUSF through AMF) may inform (e.g., with a NAS message) the UE about its confirmed role as a PEGM or PEMC. The CN may also inform the UE about any specific PIN configuration information, e.g., as shared by the PIN AF and/or verified against the user subscription. The CN may also configure the UE with a policy and configuration parameters to route PIN related data (e.g., data originated in a PINE) to the AF using pre-defined networking resources. The CN may also configure the UE with a policy determining how/whether to route data or what data to route, e.g., to/from cellular PINE devices from/to non-cellular PINE devices within the PIN. Additionally, or alternatively, some of above information may also be sent by the AF. The UE configuration/policy determining whether to route data of PINE devices implies that this configuration can be used for the authorization of the communications of a PINE device.

At stage 6, the AF may also configure then the PEGM/PEMC with other configurations, e.g., related to PIN parameters.

Some of the stages 1-6 may be included, e.g., in step 1602 in FIG. 16. They resemble a security procedure by which a UE can become PEGC/PEMC of a PIN.

At stage 7, when the UE is requested to add a PINE to the PIN or a PINE requests access to the UE PIN, the UE may:
a) setup a secure channel between UE and PINE, e.g., using a SEP, e.g., a PAKE-based SEP or a protocol based on the PINE's wireless communication technology.
b) give access to the AF based on the configuration received in stage 5 or 6 or other parameters. This access may be limited in time waiting for confirmation from the CN or AF the successful authentication procedure between PINE and AF. This access may be based on specific communication resources that can be monitored by the CN to ensure PIN subscription compliancy.

Stage 7 may correspond to Steps 1604-1607 in FIG. 16.

At stage 8, An authentication and authorization procedure may be performed between PINE and AF. This procedure may be at application layer, e.g., outside of a 3GPP specification. It might also be based on secondary authencation. Optionally or additionally or alternatively, the UE may derive a PINE key (K_PINE) e.g., from K_AF by using a KDF with such a key and, e.g., an identifier provided/scanned by the PINE. The UE may securely share this identifier with the AF that can derive the same K_PINE since it shares K_AF. The UE might also share K_PINE with the PINE. The authentication and authorization procedure might also be based on K_PINE.

At stage 9, if authentication/authorization between PINE and AF is successful in Step (8), AF informs CN (e. g, AUSF) of the new PINE, successful authentication status, and PINE requirements. AUSF adds information to UDM (UDR) about the PINE. Billing information may also be updated due to the new status of the PIN (new PINE). The CN may then inform the UE about the successful authentication of the PINE and any configuration information provided by the AF. The CN may also provide the PEGC/PEMC—by means of a NAS message—with a communication policy to route data originated in the PINE using specific communication resources or a specific identifier. Additionally or alternatively, the AF may provide the UE with the previous information (e.g., using a secure channel protected with K_AF (or a key derived from it). The UE then informs the CN, e.g., by means of a NAS message to the AMF and then to the AUSF.

At stage 10, UE may obtain or have obtained an identifier (scan QR code, enter ID, . . . ) from the PINE for identification purposes. This identifier may also have been shared in a secure way with the AF, e.g., in Step 8. This identifier, or an identifier derived from it, e.g., by means of a KDF, may be shared with the CN for identification purposes of the PINE.

At stage 11, a PINE key (K_PINE) related to the UE may be derived, e.g.:
from K_AUSF including identifier as input in KDF.
from current K_AF, including identifier as input in KDF.
provided by the AF

The AF may send PINE key to the PINE in a secure way (application layer) based on a secure link established, e.g., in Step (8) upon authentication/authorization. Additionally, or alternatively, the UE may share it. Additionally, or alternatively, CN and UE can also generate K_PINE if it is derived from K_AUSF or K_AF.

This PINE key might also be considered as a an “access token” that allows the PINE to communicate with the AF or another PINE in the PIN through the PEGC/PEMC. This “access token” ensures that only an authorized PINE can make use of the PEGC/PEMC upon authentication and/or authorization. This PINE key acting as an authorization token might be managed by the CN or AF and shared to both PINE and PEGC so that the PEGC/PINE can verify the authentication/authorization of the exchanged messages. This is advantageous since other authorization mechanisms such as filtering by MAC address do not offer strong security guarantees since the MAC addresses can be easily spoofed.

At stage 12, K_PINE may allow actions such as:
the CN/UE to send certain commands to the PINE.
the PINE to establish a secure channel with UE/CN so that the PINE can securely exchange messages with the PEGC/PEMC.
the PINE to send certain messages in a secure way to CN.
The PEGC/PEMC to send messages to the PINE, either generated locally at the PEGC/PEMC or forwarded from other PINE or the AF.

For instance,
every time a PINE sends a message to the AF over the PEGC, the PINE might attach a token based on K_PINE.
For instance, this token might be a MIC based on K_PINE, if K_PINE is a symmetric key where the MIC might be computed over the message or might be computed over a parameter that allows verifying the message freshness (e.g., an UTC based counter).
For instance, this token might be an authorization token that is verified by the PEGC/PEMC every time the PINE sends a message to the PEGC/PEMC and/or AF and/or another PINE.
For instance, this K_PINE might be used to (mutually) authenticate an IKEv2 IKE based tunnel setup between PINE and PEGC/PEMC so that all traffic exchanged is protected.
The managing entity of the PIN (e.g., a NF in the 5GS or an AF) might be in charge of of managing, generating, distributing, etc said tokens, e.g., said authorization tokens. The managing entity of the PIN might be in charge of providing the PINEs and/or PEGC/PEMC with keying materials (e.g., a symmetric-key or a private key) so that they are capable of verifying the tokens.

Some of the stages 8-12 may be included in step 1608 in FIG. 16. They resemble an authentication and authorization process of a PINE.

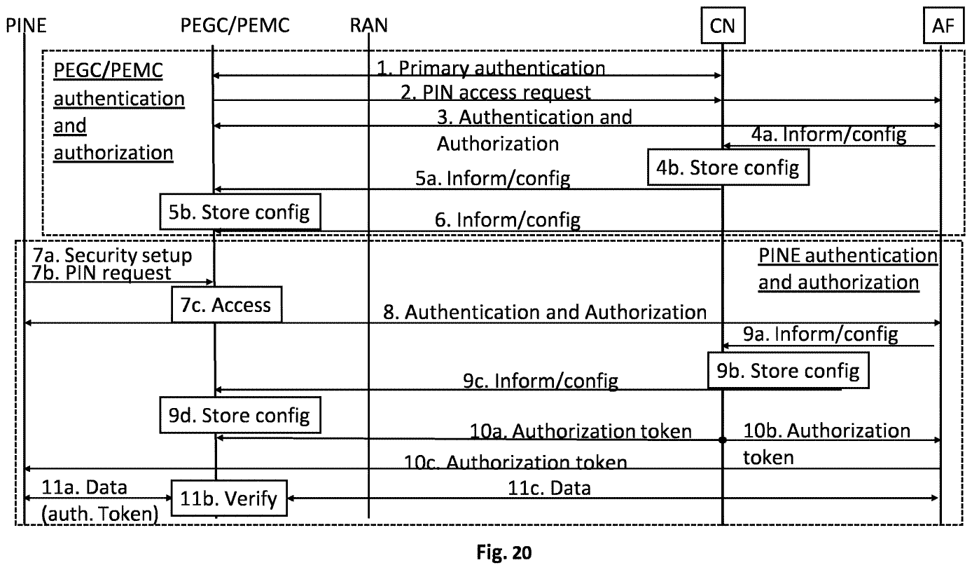

FIG. 20 schematically provides a view of above stages wherein:
in Stage 1, a primary authentication may be performed between UE (PEGC/PEMC) and CN;
in Stage 2, the UE (or an application on it) may request PIN access to the CN or to an AF through the CN. This may be through AKMA (TS 33.535) and the AnAF. In this case, K_AKMA is used to derive a K_AF for the specific UE and PIN. Alternatively, a different anchor function specific to PIN may be used, e.g., an Anchor PIN function (AnPF) in charge of PIN key and device management (PKaDMA), e.g., managing an K_PKMA. Such as K_PKMA may play a role similar to K_AKMA but be specific to the PINs a UE is member/in charge of K_PKMA might be derived in a similar way as K_AKMA, i.e., from K_AUSF, but using in the KDF as input parameters other parameters such as a bitstring identifying the PKaDMA. From K_PKMA a K_PIN can be derived, similarly as done foor K_AF, e.g., using as input an identifier identifying the PIN. This allows supporting more than a single PIN. From K_PIN or from K_PKMA it might be feasible to derive K_PINE, as explained above, a key specific to a given PINE. Having a different NF AnPF is advantageous since its PIN scope is different than that of AnAF and allows placing all PIN functionality in an isolated NF.
in Stage 3, the UE and AF may perform an authentication and authorization step. This step maybe based on keys distributed in Stage 2.
In Stage 4a, the AF may inform the CN about the outcome of Stage 3 and provide the CN with a configuration. This configuration might include information about: PIN identifier, PIN purpose, PIN elements, PIN element capabilities, communication requirements such as QoS, allowed interactions between PINE, etc. In Stage 4b, the CN may store the configuration, e.g., in the UDR or in the AnPF.
In Stage 5a, the CN may inform the UE about the outcome of Stage 4 and may provide the UE with a configuration. This configuration may be based on the configuration exchange in Stage 4 and it may include related. This configuration may include rules to enable an authentication and authorization procedure (as in Stage 8) for a PINE (e.g., as required in Stage 7c). This configuration may include an “authorization token” for specific PINEs as described above. In Stage 5b, the UE may store the configuration. This configuration received from the CN relates to communication parameters assigned to the PIN.
In Stage 6, the AF may inform the UE about the outcome of Stage 4 and may provide the UE with a configuration, e.g., keying materials such as certificates, passwords, etc. This configuration received from the AF may relate to application-related aspects assigned to the PIN by the AF.
In Stage 7a, the PINE and PEGC/PEMC may setup a secure communication channel. In Stage 7b, the PINE may send a PIN access request to the PEGC/PEMC. In Stage 7c, the PEGC/PEMC may grant (temporary) access (to the PINE), e.g., based on information received in Stage 5a, to perform Stage 8.

Stage 7a might be based on a SEP as in above embodiments, e.g., a PAKE or another protocol, such as the Device Provisioning Protocol (DPP) or Wi-Fi Easy Connect™M. The advantage of using a PAKE or the DPP is that communication links are individually protected
In Stage 8, PINE and AF may perform an authentication and authorization step.
In Stage 9a, the AF may inform the CN about the outcome of Stage 8 and may provide the CN with a configuration related to the PINE. In Stage 9b, the CN may store the configuration. In Stage 9c, the CN may inform the PEGC/PEMC about the outcome of Stage 8 and may provide the PEGC/PEMC with a configuration for the PINE. In Stage 9d, the PEGC/PEMC may store the configuration. This configuration received from the CN may relate to communication parameters assigned to the PINE.
In Stage 10, the PINE and PEGC/PEMC may receive an “authorization token” from the CN. The PINE may receive it through the AF. The goal of this “authorization token” is to ensure that only authenticated/authorized PINEs can communicated with/through the PEGC/PEMC.
In Stage 11, data may be exchanged between PINE/PEGC/PEMC authenticated and/or authorized with said authentication token.

Related to FIG. 20, stages 1-6 may correspond to the PEGC/PEMC authentication and/or authorization procedure. Stages 7-11 may correspond to the PINE authentication and/or authorization procedure. These procedures might be used together, but they might also be implemented independently. The message “PIN access request” in Stage 7b is a message that a PINE can send over different lower layers (such as Wi-Fi and PC5) to a UE serving as PEGC/PEMC. The reception of this message gives an indication to the UE of the request to join the PIN. This message might be the initial message of Stage 8 (PINE authentication and authorization). This message may advantageously include an identifier of the target PIN, e.g., an identifier related to the PIN.

The PINE might send the “PIN access request” upon reception of a “PIN announcement” message. This is a message sent/broadcasted by the UE announcing its PIN capabilities. This message might also include additional information that allows a PINE to determine whether it is the correct PIN to join. This message might include an identifier of the PINE so that the PINE knows that the UE is trying to connect to it. The PINE might send the “PIN access request” upon certain out-of-band interaction, e.g., scanning a QR code displayed by the UE.

In a variant of this step, since a PIN Element may establish a connection to the PEMC and PEGC using the local interface (PC5, WLAN or Bluetooth etc and may eventually be authorized by the PEMC to join the PIN, the PEMC/PEGC may require a policy or configuration determining/specifying one or more of the following:
the types of devices that are allowed to join the PIN,
the types of interactions (authentication with AF, communication PINE-AF, communication between PINE, etc.) between the devices (e.g., 3GPP and non-3GPP devices) that are authorized,
the conditions (time, location, context etc.) under which an interaction/communication is authorized,
the type of allowed lower layers that a PINE can use in a PIN,
the devices that are allowed to join the PIN,
the type of authentication procedures, e.g., based on 1) scope (local, end-to-end and so on), 2) type (e.g., Matter-based, DPP-based etc.) to support PINE authorization,
credentials to verify an authorized PINE,

This policy may be configured by, amongst other things:
An AF or
a NF in the 5GS or
locally in the PEGC/PEMC (e.g., by means of a UI).

In a variant of this step, the PEGC/PEMC might adjust access decisions of a PINE based on said configuration or policy.

In a variant of this step, this policy may be applied to Step 3 of Solution 8 in TR 33.882 v 0.4.0.

Stage 8 is an authentication and authorization step. This might be done based on an application layer protocol, secondary authentication, or it might also relay on a peer, pass-through authenticator and back-end authentication server architecture and using the EAP protocol similar as in Clause 6.1.1.2 in TS 33.501. The peer is the PINE, the authenticator maybe the UE (on behalf of the 5GS) and the authentication server is the AF. The peer sends EAP messages over the (wireless) communication technology between PINE and UE, e.g., EAP over LAN or EAPOL. Then the UE acting as authenticator exchanges EAP frames (e.g., directly or encapsulated in a protocol such as diameter) with the authentication server, i.e., the AF.

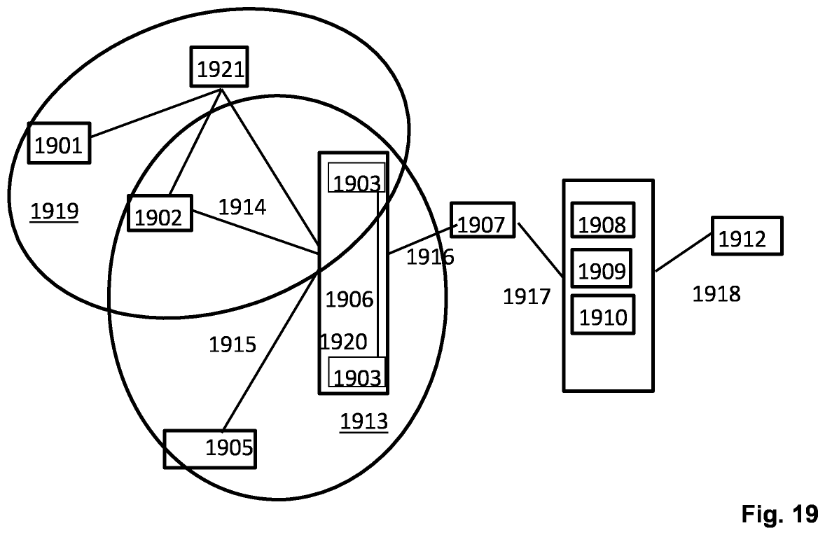

FIG. 19 schematically provides a different view of the elements in a personal IoT network (PIN), similar to FIG. 16. The elements in FIG. 19 are as follows:
Device 1901 is a non-cellular device not part of the PIN.
Device 1902 is a non-cellular device part of the PIN.
Device 1903 is a cellular device capable of managing the PIN, i.e., PEMC.
Device 1904 is a cellular device capable of acting as a gateway of the PIN, i.e., PEGC.
Device 1905 is a cellular PINE device part of the PIN.
Device 1906 is a communication link between devices 1903 and 1904
Device 1907 is a cellular access device, e.g., gNB.
Device 1908 is the AMF in the CN.
Device 1909 is the AUSF in the CN.
Device 1910 is a NF in the CN such as UDM, UDR, AKMA, UPF, PCF, NEF, AF, etc. in the CN.
Element 1911 Is the Cn.
Element 1912 is an AF in charge of managing the PIN.
Network 1913 is the PIN including 1902, 1905 and 1920.
Link 1914 is a non-cellular communication link between 1920 and 1902.
Link 1915 is a cellular communication link (e.g., PC5) between 1905 and 1920.
Link 1916 is a cellular communication link (e.g., Uu) between 1920 and 1907.
Link 1917 is a communication link interfacing 1907 and 1911 or a network function in it.
Link 1918 is a communication link between 1911 and 1912.
Network 1919 is a non-cellular network.
Device 1920 is a device including the functionalities of 1905 and 1906.
Device 1921 is a non-cellular device managing or giving access to the non-cellular network.

In FIG. 19, it may be seen that device 1920 is part of 1919,—for example, it has joined that network and has credentials, such as a security key. Since device 1920 is part of network 1919, device 1920 can also communicate with device 1902 (part of network 1919) as well) over link 1914.

Device 1902 has also joined network 1913 where device 1920 has managed the authentication/authorization procedure on behalf of elements 1911 or 1912 in order to access networks 1913 and links 1916 or 1915. When device 1902 needs to communicate with device 1905, or vice versa, communications are routed through device 1920 where device 1920 received from element 1911 or 1912 a policy determining which communication flows are allowed to be rerouted. In FIG. 19, device 1921 manages network 1919 and device 1920 is a part of network 1919. This implies that device 1920 also joins network 1919 first. Alternatively, device 1920 may include the functionalities of device 1921. In this case, device 1921 handles which devices join network 1919 first and then which of those devices join network 1913. When a new device is accepted in network 1913 by device 1920, device 1920 may inform other devices about the newly joined device.

In general, there is a method and apparatus, which can be implemented in a device, allowing one or more of the following actions:
Receiving a configuration for a first device (e.g., PEGC/PEMC or cellular based PINE) from an AF executed on a third communication device;
Performing a SEP (e.g., DPP, PAKE-based) between the first device and a second device (e.g., non-cellular PINE) allowing the initial establishment of a secure and authenticated channel with the second device through a first communication link, e.g., Wi-Fi;
Routing further authentication messages through a first device between the second device and the NF/AF on the third communication device where the communication between the first device and third device goes through a second cellular backhaul communication link;
Receiving an authorization confirmation and/or authorization configuration at the first device from the third device determining the access rights of the second communication device to at least one of:
   accessing the first device network;
   accessing the second cellular backhaul communication link (e.g., Uu interface);
   communicating with a fourth device (e.g., cellular PINE) connected to the first device through a cellular communication link (e.g., sidelink/PC5).

In at least some of the above embodiments, the PAKE may be SPAKE2+ or a different PAKE, either balanced or augmented.

In at least some of the above embodiments, the password may be a shared symmetric key.

In at least some of the above embodiments, the password (PWD) may be a concatenation of (short) shared symmetric keys and metadata determining a validity of the password and communication link, e.g., how long it is valid and which access rights it involves. For instance:

Pwd=K|Metadata,

wherein the metadata may include an access policy or access roles. It is to be noted that this approach can be used with other PAKE schemes. In some cases, it may be required to apply a function on the input K|Metadata, e.g., the PBKDF or a different function.

In some of the embodiments, the “metadata” value may be the root of a Merkle tree computed from a set of leaves where each leaf includes some information about the device. This allows the device to disclose only part of its data in its leaves. The device may only exchange the data once the secure connection is established.

This proposed technique has advantages compared with alternative techniques that can be used to grant authentication and authorized. For instance:
if a conventional access token is used, initiator and responder may first establish a secure channel, e.g., using the Diffie-Hellman (DH) algorithm (which is a key-exchange protocol that enables two parties communicating over a public channel to establish a mutual secret without it being transmitted over the Internet). Then, the initiator may send an access token to the responder. The access token may contain information stating the access rights of the initiator and may be signed by an issuing party trusted by the responder. This scheme may require digital signatures, and it may still be prone to man-in-the-middle (MitM) attacks. In contrast, the proposed scheme is resistant to MitM attacks and does not require digital signatures.
If initiator and responder rely on PWD=K|Metadata to perform symmetric-key mutual authentication and key agreement protocol. This may expose which passwords are assigned to which devices and therefore their identities. Furthermore, if the same password is assigned to a third device as initiator and responder, the third device can access the communication link between initiator and responder.

In some of the above embodiments, one of the messages in the preamble or PAKE may include a password hint indicating the receiving party which password or password-based information (e.g., referring to an augmented PAKE, e.g., in SPAKE2+ this password-based information refers to the verification value pair L and w0) to use. In this case, if a password has been configured, e.g., by a central authority such as the 5G CN, or if the user is not sure which password to use, or if the user is not sure which QR (containing the password) to scan, then the device may use the password hint to indicate it so that the other party knows which password to retrieve and use to authenticate the other party after executing the PAKE. Successful authentication may imply verification of the password and metadata. For instance, the password hint may be obtained as a function, e.g., a KDF or a hash, of the password and additional parameters such as a salt, a nonce, a counter, etc., as follows:
PWD_Hint=KDF (PWD|additional parameters)
PWD_Hint=Hash (PWD|additional parameters)
PWD_Hint=HMAC (PWD|additional parameters), or the password hint may be a bit string randomly generated and associated to the password.

In at least some of the above embodiments, the password may comprise at least two passwords. A first password provided by a central management entity to both communicating parties authorizing the establishment of the communication channel and a second password entered by the user enabling the user to authorize the setup of the secure communication channel.

PWD=PWD1|PWD2

In some of the above embodiments, two PAKEs may be executed in parallel, a first password PWD1 used in a balanced PAKE where the password may be determined, e.g., by the users involved, and a second password PAWD2 used in an augmented PAKE where the password may be determined by the network/system management. Executing the PAKEs in parallel means that the PAKE messages may be sent simultaneously reducing the number of round trips. In similar PAKEs, steps or operations can be combined. This has the advantage that users can enter their own password to authorize the communication while the network/system can distribute in an initial configuration phase a password to the involved devices, e.g., to authorize the communication link, e.g., a first device (e.g., initiator) to communicate with a second device (the responder).

In at least some of the above embodiments, a central managing party, e.g., the 5GS, may support authorization of the UE as a UE-to-UE relay in the UE-to-UE relay scenario.

In at least some of the above embodiments, a central managing party, e.g., the 5GS may support authorization of the UE as a Source-UE or a Target UE in the UE-to-UE relay scenario.

The fact that a PAKE may be augmented is a useful feature since if the responder or verifier is compromised, the best that an attacker can do to impersonate the initiator or client is to mount an offline attack. This allows the central managing party to deploy a password PWD as above, i.e., K|Metadata that can be used by the client to show its authenticity and authorization, e.g., to show it is authorized to act as a Source-UE to the target UE. A central managing party may create, e.g., two types of passwords/keys: K_S and K_T. K_S may be provided to UEs that can act as source only, K_T may be provided to UEs that can act as target only. The counter part of those passwords/keys may be provided to the other party, e.g., a target device that can only act as target is provided with a function of a bitstring derived from K_S (and metadata) that is difficult to invert (in a security meaning). It is to be noted that if a balanced PAKE is used, then a central managing party may also be able to assign a password/key to a pair of devices authorizing them to communicate with each other.

In some of the above embodiments, one of the devices, e.g., the responder device, may also retrieve or request the password (or password-based value in an augmented PAKE) from such a central authority, e.g., upon reception of a preamble message containing a password hint. To this end, the device may need to share with the central authority certain parameters, e.g., a password hint or exchanged parameters to be used in the PBKDF. In the case of a password-based value in an augmented PAKE, this has the advantage that responder device may perform the subsequent key exchange, e.g., PAKE, with the initiator device on behalf of the central authority without having access to the password. Since the password-based value may depend on exchanged parameters to be used in the PBKDF, this value has limited validity. Only if the procedure is successful, the responder may allow the initiator to perform certain actions, e.g., have access to networking resources, the 5G core network, or an application function accessible through the 5G system.

In some of the above embodiments, an exchanged parameter to be used in a key derivation function may be (the least significant bits of) a UTC-based counter.

To summarize, methods and devices for setting up a secure communication channel with an improved key exchange for an SEP have been described.

While the invention has been illustrated and described in detail in the drawings and foregoing description, such illustration and description are to be considered illustrative or exemplary and not restrictive. It should be understood that embodiments may be combined and that features of any one of the embodiments may be used in combination with another embodiment. The invention is not limited to the disclosed embodiments. The proposed enhancements for SEPs (such as SPAKE2+ or SPAKE or PAKEs in general) can be implemented in all types of wired or wireless networks, e.g., it can be applied to devices or applications communicating using cellular wireless communication standards, specifically the 3^{rd} Generation Partnership Project (3GPP) 5G and New Radio (NR) specifications. The communication devices or applications can be different types of devices, e.g., mobile phones, smart watches, smart tags for location tracking and logistics, commissioning devices, applications or tools, vehicles (for vehicle-to-vehicle (V2V) communication or more general vehicle-to-everything (V2X) communication), V2X devices, IoT hubs, IoT devices, including low-power medical sensors for health monitoring, medical (emergency) diagnosis and treatment devices, for hospital use or first-responder use, virtual reality (VR) headsets, etc., and may be used e.g. in personal IoT networks (PIN) and/or ProSe with focus on UE-to-UE.

Furthermore, the proposed enhancements for SPAKE2+, or PAKEs in general, or other SEPs can be used in applications such as Wi-Fi, cloud access, browser synchronization, e-passports, in the Thread network protocol for IoT devices, in PAKE standards developed by standard developing organizations (SDOs) such as IEEE, ISO/IEC, or IETF, in IEEE 802.11s protocols and WiFi protected access (e.g., WPA3 e.g. in connection with Simultaneous Authentication of Equals (SAE)), in protocols for transport layer security (e.g., TLS 1.3) or internet key exchange (e.g., IKEv 2).

Although ProSe relay and sidelink communication have been mentioned, the present invention also applies to other types of relay devices, e.g., Proximity Services involving direct communication between two UEs, e.g., Proximity Services involving UE-to-Network relay, ranging services, e.g., (smart) repeater devices, Integrated Access and Backhaul (IAB) nodes, or Wi-Fi Mesh APs. Furthermore, the techniques mentioned here can also be applied to other procedures, e.g., to address the key issues identified for ranging procedures in TR 33.893v0.2.0 .

Furthermore, the invention can be applied in medical applications or connected healthcare in which multiple wireless (e.g. 4G/5G) connected sensor or actuator nodes participate, in medical applications or connected healthcare in which a wireless (e.g. 4G/5G) connected equipment consumes or generates occasionally a continuous data stream of a certain average data rate, for example video, ultrasound, X-Ray, Computed Tomography (CT) imaging devices, real-time patient sensors, audio or voice or video streaming devices used by medical staff, in general IoT applications involving wireless, mobile or stationary, sensor or actuator nodes (e.g. smart city, logistics, farming, etc.), in emergency services and critical communication applications, in V2X systems, in systems for improved coverage for 5G cellular networks using high-frequency (e.g. mmWave) RF, and any other application areas of 5G communication where relaying is used.

Other variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. The foregoing description details certain embodiments of the invention. It will be appreciated, however, that no matter how detailed the foregoing appears in the text, the invention may be practiced in many ways, and is therefore not limited to the embodiments disclosed. It should be noted that the use of particular terminology when describing certain features or aspects of the invention should not be taken to imply that the terminology is being re-defined herein to be restricted to include any specific characteristics of the features or aspects of the invention with which that terminology is associated.

Furthermore, in those instances where a convention analogous to “at least one of A, B, and C, etc.” is used, in general such a construction is intended in the sense one having skill in the art would understand the convention, e.g., “a system having at least one of A, B, and C” would include but not be limited to systems that have A alone, B alone, C alone, A and B together, A and C together, B and C together, and/or A, B, and C together, etc. In those instances where a convention analogous to “at least one of A, B, or C, etc.” is used, in general such a construction is intended in the sense one having skill in the art would understand the convention, e.g., “a system having at least one of A, B, or C” would include but not be limited to systems that have A alone, B alone, C alone, A and B together, A and C together, B and C together, and/or A, B, and C together, etc. It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.

A single unit or device may fulfill the functions of several items recited in the claims. The mere fact that certain measures are recited in mutually different dependent claims does not indicate that a combination of these measures cannot be used to advantage.

The described operations like those indicated in FIGS. 1 to 3 and 5 to 13 can be implemented as program code means of a computer program and/or as dedicated hardware of the commissioning device or luminaire device, respectively. The computer program may be stored and/or distributed on a suitable medium, such as an optical storage medium or a solid-state medium, supplied together with or as part of other hardware, but may also be distributed in other forms, such as via the Internet or other wired or wireless telecommunication systems.

1. An apparatus for controlling a security establishment process between a first communication device (A) and a second communication device (B) over a transmission link, wherein the apparatus is adapted to:
receive from the first communication device (A) a preamble message (Pre_AB) including a security establishment identifier (ID_A); and
allocate the security establishment identifier (ID_A) received from the first communication device (A) as peer session identifier (401) for subsequent messages;
wherein the apparatus is further adapted to limit the number of first preamble messages allowed per unit of time.

2. The apparatus of claim 1, wherein the first communication device (A) comprises a commissioning tool configured to use the security establishment process to interact with the second communication device (B) in order to perform at least one selected from the group of commissioning, configuring, authenticating and authorizing.

3. The apparatus of claim 1, wherein the second communication device (B) is comprised in a medical device or a personal healthcare device or a smart home device.

4. The apparatus of claim 1, wherein the apparatus is configured to run a password authenticated key exchange protocol to mutually authenticate the first and second communication devices (A, B) to each other and establish a secret.

5. The apparatus of claim 1, wherein preamble messages are exchanged between the first and second communication devices (A, B) or between the first communication device (A) or the second communication device (B) and a relay device in an initial device discovery phase.

6. The apparatus of claim 1, wherein a single preamble message is exchanged between the first and second communication devices (A, B).

7. The apparatus of claim 1, wherein a content of a preamble message is included in a discovery message and wherein the first security establishment message is included in a direct communication request message.

8. The apparatus of claim 6, wherein the apparatus is further adapted to run a password authenticated key exchange protocol to mutually authenticate the first and second communication devices (A, B) to each other and establish a secret over a first wireless communication protocol and authorize the first communication device (A) to access communication resources of a second communication link.

9. The apparatus of claim 8, wherein the second communication link uses a cellular communication protocol.

10. A communication device comprising an apparatus according to claims 1.

11. A method of controlling a security establishment process between a first communication device (A) and a communication device (B) over a transmission link, wherein the method comprises:
receiving from the first communication device (A) a preamble message (Pre_AB) including a security establishment identifier (ID_A); and
allocating the security establishment identifier (ID_A) received from the first communication device (A) as peer session identifier (401) for subsequent messages;
wherein the method further comprises limiting the number of first preamble messages allowed per unit of time.

12. A computer program product comprising code means for producing the steps of claim 11 when run on a computer device.

13. A system comprising two or more communication devices including a communication device according to claim 10.

14. The apparatus of claim 1, adapted to
receive from the first communication device (A), in the preamble messge, a previous random value (R, B′) previously sent to the first communication device (A); and
wherein the allocating of the security esablishment identifer (ID A) is dependent on a random value (R, B) of the second communication device (B) being equal to the previous random value (R, B′).
